(19) United States
(12) Patent Application Publication
(10) Pub. No.: US 2023/0234997 A1
(43) Pub. Date: Jul. 27, 2023

(54) Compositions and Methods for the Treatment of Synucleinopathies

(71) Applicant: SOLA BIOSCIENCES LLC, Natick, MA (US)

(72) Inventors: Akinori Hishiya, Belmont, MA (US); Keizo Koya, Chestnut Hill, MA (US)

(21) Appl. No.: 18/007,978

(22) PCT Filed: Jun. 4, 2021
(86) PCT No.: PCT/US21/35958

§ 371 Date: Dec. 2, 2022

Related U.S. Application Data
(60) Provisional application No. 63/035,297, filed on Jun. 5, 2020.

(51) Int. Cl. C07K 14/47; C12N 15/86; A61P 3/00

(52) U.S. Cl. CPC C07K 14/47; C12N 15/86; A61P 3/00; C12N 2750/14143; C07K 2319/74

(57) ABSTRACT

A novel class of fusion proteins to recruit a cell's innate chaperone mechanism, specifically the Hsp70-mediated system, to specifically reduce α-synuclein-mediated protein aggregation and associated proteopathies is disclosed.

### CROSS REFERENCE TO RELATED APPLICATION

This application claims priority under 35 U.S.C. § 119(e) to the United States Provisional Patent Application 63/035,297 filed Jun. 5, 2020. The entire contents of the aforementioned application is hereby incorporated by reference in its entirety.

### SEQUENCE LISTING

This application incorporates by reference in its entirety the Sequence Listing entitled “269548-493385_ST25.txt” (68 KB), which was created on Jun. 4, 2021 at 1:09 PM, and filed electronically herewith.

### TECHNICAL FIELD

New fusion proteins comprising one or more J domains, α-synuclein-binding domain, linkers, targeting reagents, epitopes, cell-penetrating agents, and combinations thereof, are described and claimed. In addition, the present disclosure describes the use of these novel fusion proteins, and their recruitment of innate innate chaperone mechanisms, e.g., the Hsp70-mediated system, to specifically reduce α-synuclein-mediated protein aggregation and associated proteopathies, are likewise described and claimed.

### BACKGROUND

All proteins expressed within a cell need to correctly fold into their intended structures in order to function properly. A growing number of diseases and disorders are shown to be associated with inappropriate folding of proteins and/or inappropriate deposition and aggregation of proteins and lipoproteins as well as infectious proteinaceous substances. Also known as a conformational disease or proteopathy, examples of diseases caused by misfolding include Alzheimer's disease (AD), amyotrophic lateral sclerosis (ALS), and frontotemporal lobar dementia (FTLD). The mutant protein aggregates in cells causing typical cytotoxic cellular inclusion bodies.

A wide variety of neurodegenerative diseases are characterized pathologically by the accumulation of intracellular or extracellular protein aggregates composed of amyloid fibrils (Forman et al., (2004) Nat Med. 10:1055-1063). For example, the pathology of Alzheimer's disease (AD) is defined by senile plaques and neurofibrillary tangles composed of β-amyloid and microtubule-associated protein tau, respectively.

Parkinson's disease (PD) is the second most common neurodegenerative disorder after Alzheimer's disease (AD). More than 1% of people over 60 years old suffer the disease with over 1 million patients in the US alone. PD patients experience slowness of movement, rigidity, tremors, difficulty with balance and variable manifestation of dementia in about 40% of PD patients, some of whom develop an AD-like dementia in the latter stages of the disease. The main pathological features of PD are the loss of the dopaminergic neurons in the substantia nigra and the presence of abnormal protein aggregates that form filamentous inclusions in neuronal cytoplasm, termed Lewy bodies (LBs) or Lewy neurites (nerve fibers) (LN) in PD brains (Galvin et al., (2001) Arch Neurol 58, 186-190; Lang and Lozano, (1998) N Engl J Med 339, 1044-1053; Lang and Lozano, (1998) N Engl J Med 339, 1130-1143). The majority of PD is sporadic. Familial forms of PD represent about 10% of all cases and could be either autosomal recessive or autosomal dominant, suggesting a complicated etiology of the disorder (Reviewed, Lang & Lozano) (Lang and Lozano, (1998) N Engl J Med 339, 1044-1053; Lang and Lozano, (1998) N Engl J Med 339, 1130-1143). In addition, environmental factors also play important roles in the development of PD (Reviewed by Di Monte, 2003) (Di Monte et al., (2003) Lancet Neurol 2, 531-538).

The discoveries of genetic linkages for PD to several loci provide promises to identify genetic factors that contribute to the pathogenesis of the disease. Studies of patients with rare, dominantly inherited variants of PD in a family originated from the south of Italy linked the disorder to a locus on chromosome 4q21-23 (park 1), where a previously identified gene called α-synuclein (SNCA) was located (lakes et al., (1994) FEBS Lett 345, 27-32; Shibazaki et al., (1995) Cytogenet Cell Genet 71, 54-55; Polymeropoulos et al., (1996) Science 274, 1197-1199). Direct evidence for the involvement of α-synuclein in familial PD was provided by identification of a missense mutation in the gene leading to an A-T substitution at amino acid 53 in this Italian-American family and thereafter three unrelated Greek families (Polymeropoulos et al., (1997) Science 276, 2045-2047). Subsequently, a second mutation at position 30 of α-synuclein gene changing alanine to proline in a German family was identified (Kruger et al., (1998) Nat Genet 18, 106-108). Immunohistochemical examination of LBs showed that LBs contain many different proteins. The most consistently described proteins are neurofilament, ubiquitin and alpha-synuclein (α-synuclein) (Takeda et al., (1998) Am J Pathol 152, 367-372; Gai et al., Brain 118 (Pt 6), 1447-1459). The large juxtanuclear aggregates of misfolded α-synuclein is predominantly present in Lewy bodies as well as in dystrophic Lewy neurites (LNs) of PD brains (Dickson, (2012) Cold Spring Harb Perspect Med 2; Stefanis, (2012) Cold Spring Harb Perspect Med 2, a009399; Lashuel et al., (2013) Nat Rev Neurosci 14, 38-48), suggesting misfolding of α-synuclein as the main culprit in the pathogenesis of neurodegenerative diseases associated with aggregated α-synuclein accumulation (Baba et al., (1998) Ami Pathol 152, 879-884; Kalia et al., (2013) Ann Neurol 73, 155-169).

The α-synuclein gene encodes a 140 amino acid protein of ^{˜}19 kDa. Although the functions of α-synuclein are not completely clear, there is evidence suggesting that α-synuclein has a role in neurotransmitter release (Liu et al., (2004) EMBO J 23, 4506-4516; Chandra et al., (2005) Cell 123, 383-396; Fortin et al., (2005) J Neurosci 25, 10913-10921). α-synuclein is highly expressed throughout mammalian brain but enriched in pre-synaptic nerve terminals, associated with membranes and vesicular structures. Mice have a functional homologue of α-synuclein. Homozygous knockout of mouse α-synuclein gene is viable, fertile and almost normal (Abeliovich et al., (2000) Neuron 25, 239-252). α-synuclein−/−mice exhibit an increased DA release with paired stimuli, a reduction in striatal DA, and an attenuation of DA-dependent locomotor response to amphetamine, suggesting that α-synuclein is a regulator of DA neurotransmission. Mice with α-synuclein over-expression were apparently normal although there was a significant degeneration of DA nerve terminals in the mice lines with high expression of human α-synuclein, but no loss of DA neurons was observed (Masliah and Rockenstein, (2000) J Neural Transm Suppl 59, 175-183). Over-expression of the A53T α-synuclein mutant induced substantial neurodegeneration (Lee et al., (2002) Proc Natl Acad Sci USA 99, 8968-8973; Giasson et al., (2002) Neuron 34, 521-533; Dawson et al., (2002) Neuron 35, 219-222). Progressive loss of DA neurons in rats that overexpress wild-type and mutant α-synuclein mediated with adeno-associated virus (AAV) suggests that PD models could be used for testing PD treatments (Kirik et al., (2002) Neuron 35, 219-222).

Currently, there is no prevention or cure for PD but only symptomatic treatments, such as medication and surgical therapy. Degeneration of dopaminergic nigral neurons leads to loss of dopaminergic projections to the striatum, which represents the primary defect of neurochemical pathways in PD. As such, one treatment for PD is the replacement of dopamine (DA), in which patients take the drug levodopa. Levodopa is converted into the dopamine by aromatic L-amino acid decarboxylase (AADC). However, the benefit is temporary because of numerous side effects (Reviewed by Nutt 2003) (Nutt, (2003) Exp Neurol 184, 9-13). In addition, less AADC is available as disease progresses so that patients may experience increased off-time such as stiffness, rigidity and cramping. Therefore, a substantial need exists for the development of new treatments for this devastating disease.

The heat shock 70 kDa proteins (referred to herein as “Hsp70s”) constitute a ubiquitous class of chaperone proteins in the cells of a wide variety of species (Tavaria et al., (1996) Cell Stress Chaperones 1, 23-28). Hsp70 requires assistant proteins called co-chaperone proteins, such as J domain proteins and nucleotide exchange factors (NEFs) (Hartl et al., (2009) Nat Struct Mol Biol 16, 574-581), in order to function. In the current model of Hsp70 chaperone machinery for folding proteins, Hsp70 cycles between ATP- and ADP-bound states, and a J domain protein binds to another protein in need of folding or refolding (referred to as a “client protein”), interacting with the ATP-bound form of Hsp70 (Hsp70—ATP) (Young (2010) Biochem Cell Biol 88, 291-300; Mayer, (2010) Mol Cell 39, 321-331). Binding of the J domain protein-client complex to Hsp70-ATP stimulates ATP hydrolysis, which causes a conformational change in the Hsp70 protein, closing a helical lid and, thereby, stabilizing the interaction between the client protein with Hsp70-ADP, as well as eliciting the release of the J domain protein that is then free to bind to another client protein.

Therefore, according to this model, J domain proteins play a critical role within the Hsp70 machinery by acting as a bridge, and facilitating the capture and submission of a wide variety of client proteins into the Hsp70 machinery to promote folding or refolding into the proper conformation (Kampinga & Craig (2010) Nat Rev Mol Cell Biol 11, 579-592). The J domain family is widely conserved in species ranging from prokaryotes (DnaJ protein) to eukaryotes (Hsp40 protein family). The J domain (about 60-80 aa) is composed of four helices: I, II, III, and IV. Helices II and III are connected via a flexible loop containing an “HPD motif”, which is highly conserved across J domains and thought to be critical for activity (Tsai & Douglas, (1996) J Biol Chem 271, 9347-9354). Mutations within the HPD sequence has been found to abolish J domain function.

Given the context provided above for proteopathies such as PD, it seems clear that reducing the level of misfolded proteins could serve as a means to treat, prevent or otherwise ameliorate the symptoms of these devastating disorders and that, recruitment of a cell's innate ability to repair protein misfolding would be a logical choice to pursue.

### SUMMARY OF THE INVENTION

The inventors have developed a novel class of fusion proteins to recruit a cell's innate chaperone mechanism, specifically the Hsp70-mediated system, to specifically reduce α-synuclein mediated protein misfolding. Unlike in previous studies by the inventors using fusion proteins comprising fragments of a Hsp40 protein (also called J proteins), a co-chaperone that interacts with Hsp70, to enhance protein secretion and expression, the present study employs J domain-containing fusion proteins for the purpose of reducing protein misfolding and cytotoxicity caused by α-synuclein proteins. In this context, the inventors have made the surprising discovery that the elements of J domain required for function is quite distinct from use of J domains in enhancing protein expression and secretion, demonstrating a distinct mechanism for the mode of action of the present fusion proteins. The fusion proteins described herein comprise a J domain and a domain that has affinity for α-synuclein. The presence of the α-synuclein-binding domain within the fusion protein results in specific reduction in misfolding of α-synuclein proteins.
E1. Therefore, in a first aspect, disclosed herein is an isolated fusion protein comprising a J domain of a J protein and a α-synuclein-binding domain.
E2. The fusion protein of E1, wherein the J domain of a J protein is of eukaryotic origin.
E3. The fusion protein of any one of E1-E2, wherein the J domain of a J protein is of human origin.
E4. The fusion protein of any one of E1-E3, wherein the J domain of a J protein is cytosolically localized.
E5. The fusion protein of any one of E1-E4, wherein the J domain of a J protein is selected from the group consisting of SEQ ID Nos: 1-50.
E6. The fusion protein of any one of E1-E5, wherein the J domain comprises the sequence selected from the group consisting of SEQ ID NOs: 1, 5, 6, 10, 16, 24, 25, 31 and 49.
E7. The fusion protein of any one of E1-E6, wherein the J domain comprises the sequence of SEQ ID NO: 5.
E8. The fusion protein of any one of E1-E6, wherein the J domain comprises the sequence of SEQ ID NO: 10.
E9. The fusion protein of any one of E1-E6, wherein the J domain comprises the sequence of SEQ ID NO: 16.
E10. The fusion protein of any one of E1-E6, wherein the J domain comprises the sequence of SEQ ID NO: 25.
E1l. The fusion protein of any one of E1-E6, wherein the J domain comprises the sequence of SEQ ID NO: 31.
E12. The fusion protein of any one of E1-E1l, wherein the α-synuclein-binding domain has a K_{D} for α-synuclein of 1 μM or less, for example, 300 nM or less, 100 nM or less, 30 nM or less, 10 nM or less when measured using an ELISA assay.
E13. The fusion protein of any one of E1-E12, wherein the α-synuclein-binding domain comprises the sequence selected from the group consisting of SEQ ID NOs: 51-65.
E14. The fusion protein of any one of E1-E13, wherein the α-synuclein-binding domain comprises the sequence of SEQ ID NO:51.
E15. The fusion protein of any one of E1-E13, wherein the α-synuclein-binding domain comprises the sequence of SEQ ID NO:52.
E16. The fusion protein of any one of E1-E13, wherein the α-synuclein-binding domain comprises the sequence of SEQ ID NO:63.
E17. The fusion protein of any one of E1-E13, wherein the α-synuclein-binding domain comprises the sequence of SEQ ID NO:64.
E18. The fusion protein of any one of E1-E17, comprising a plurality of α-synuclein-binding domains.
E19. The fusion protein of any one of E1-E18, consisting of two α-synuclein-binding domains.
E20. The fusion protein of any one of E1-E19, consisting of three α-synuclein-binding domains.
E21. The fusion protein of any one of E1-E20, comprising one of the following constructs:

| | |
|---|---|
| a. | DNAJ-X-S, |
| b. | DNAJ-X-S-X-S, |
| C. | DNAJ-X-S-X-S-X-S, |
| d. | S-X-DNAJ, |
| e. | S-X-S-X-DNAJ, |
| f. | S-X-S-X-S-X-DNAJ, |
| g. | S-X-DNAJ-X-S, |
| h. | S-X-DNAJ-X-S-X-S, |
| i. | S-X-S-X-DNAJ-X-S-X-S-X-S, |
| j. | S-X-S-X-S-X-DNAJ-X-S, |
| k. | S-X-S-X-S-X-DNAJ-X-S-X-S, |
| l. | S-X-S-X-S-X-DNAJ-X-S-X-S-X-S, |
| m. | DnaJ-X-DnaJ-X-X-X-S, |
| n. | S-X-DnaJ-X-DnaJ, |
| | and |
| o. | S-X-S-X-DnaJ-X-DnaJ, |

wherein,
S is a α-synuclein-binding domain,
DNAJ is a J domain of a J protein, and
X is an optional linker.
E22. The fusion protein of any one of E1-E21, wherein the fusion protein comprises the J domain sequence of SEQ ID NO: 5 and the α-synuclein-binding domain sequence of SEQ ID NO: 51.
E23. The fusion protein of any one of E1-E22, wherein the fusion protein comprises the J domain sequence of SEQ ID NO: 5 and two copies of the α-synuclein-binding domain sequence of SEQ ID NO: 51.
E24. The fusion protein of any one of E1-E23, wherein the fusion protein comprises the sequence selected from the group consisting of SEQ ID NOs: 88, 90-96, 98-100.
E25. The fusion protein of any one of E1-E24, wherein the fusion protein comprises the sequence of SEQ ID NO: 88.
E26. The fusion protein of any one of E1-E24, wherein the fusion protein comprises the sequence of SEQ ID NO: 90.
E27. The fusion protein of any one of E1-E24, wherein the fusion protein comprises the sequence of SEQ ID NO: 99.
E28. The fusion protein of any one of E1-E24, wherein the fusion protein comprises the sequence of SEQ ID NO: 100.
E29. The fusion protein of any one of E1-E28, further comprising a targeting reagent.
E30. The fusion protein of any one of E1-E29, further comprising an epitope.
E31. The fusion protein of E30, wherein the epitope is a polypeptide selected from the group consisting of SEQ ID NOs: 77-83.
E32. The fusion protein of any one of E1-E31, further comprising a cell-penetrating agent.
E33. The fusion protein of E32, wherein the cell-penetrating agent comprises a peptide sequence selected from the group consisting of SEQ ID NOs:84-87.
E34. The fusion protein of any one of E1-E33, further comprising a signal sequence.
E35. The fusion protein of E34, wherein the signal sequence comprises the peptide sequence selected from the group consisting of SEQ ID NOs: 102-104.
E36. The fusion protein of any one of E1-E35, which is capable of reducing misfolding of α-synuclein proteins in a cell.
E37. The fusion protein of any one of E1-E36, which is capable of reducing phosphorylated α-synuclein proteins in a cell.
E38. The fusion protein of any one of E1-E37, which is capable of reducing secretion of α-synuclein proteins.
E39. The fusion protein of any one of E1-E38, which is capable of reducing α-synuclein-mediated cytotoxicity.
E40. A nucleic acid sequence encoding the fusion protein of any one of E1-E39.
E41. The nucleic acid sequence of E40, wherein said nucleic acid is DNA.
E42. The nucleic acid sequence of any one of E41, wherein said nucleic acid is RNA.
E43. The nucleic acid sequence of any one of E40-E42, wherein said nucleic acid comprises at least one modified nucleic acid.
E44. The nucleic acid sequence of any one of E40-E43, further comprising a promoter region, 5′ UTR, 3′ UTR such as poly(A) signal.
E45. The nucleic acid sequence of E44, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.
E46. A vector comprising the nucleic acid sequence of any one of E40-E45.
E47. The vector of E46, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.
E48. A virus particle comprising a capsid and the vector of E46 or E47.
E49. The virus particle of E48, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-Dian AAV capsid mutant, an AAV hybrid serotype, an organ-tropic AAV, a cardiotropic AAV, and a cardiotropic AAVM41 mutant.
E50. The virus particle of E48 or E49, wherein the capsid is selected from the group consisting of AAV2, AAV5, AAV8, AAV9 and AAVrh10.
E51. The virus particle of any one of E48-E50, wherein the capsid is AAV2.
E52. The virus particle of any one of E48-E50, wherein the capsid is AAV5.
E53. The virus particle of any one of E48-E50, wherein the capsid is AAV8.
E54. The virus particle of any one of E48-E50, wherein the capsid is AAV9.
E55. The virus particle of any one of E48-E50, wherein the capsid is AAV rh10.
E56. A pharmaceutical composition comprising an agent selected from the group consisting of the fusion protein of any one of E1-E39, a cell expressing the fusion protein of E1-E39, the nucleic acid of any one of E40-E45, the vector of any one of E46-E47, the virus particle of any one of E48-E55, and a pharmaceutically acceptable carrier or excipient.
E57. A method of reducing toxicity of a α-synuclein protein in a cell, comprising contacting said cell with an effective amount of one or more agents selected from the group consisting of fusion protein of any one of E1-E39, a cell expressing the fusion protein of E1-E39, the nucleic acid of any one of E40-E45, the vector of any one of E46-E47, the virus particle of any one of E48-E55, and the pharmaceutical composition of E56.
E58. The method of E57, wherein the cell is in a subject.
E59. The method of any one of E57-E58, wherein the subject is a human.
E60. The method of any one of E57-E59, wherein the cell is a cell of the central nervous system.
E61. The method of any one of E57-E60, wherein subject is identified as having a α-synuclein disease.
E62. The method of E61, wherein the α-synuclein disease is selected from the group consisting of PD, dementia with Lewy bodies, multiple system atrophy, and diseases related to abnormal accumulation of aggregated α-synuclein proteins (synucleinopathies).
E63. The method of E61 or E62, wherein the α-synuclein disease is PD.
E64. The method of any one of E57-E63, wherein there is a reduction in the amount of misfolded α-synuclein protein in the cell when compared with a control cell.
E65. A method of treating, preventing, or delaying the progression of a α-synuclein disease in a subject in need thereof, the method comprising administering an effective amount of one or more agents selected from the group consisting of with the fusion protein of any one of E1-E39, a cell expressing the fusion protein of E1-E39, the nucleic acid of any one of E40-E45, the vector of any one of E46-E47, the virus particle of any one of E48-E55, and the pharmaceutical composition of E56.
E66. The method of E65, wherein the α-synuclein disease is selected from the group consisting of PD, dementia with Lewy bodies, multiple system atrophy, and diseases related to abnormal accumulation of aggregated α-synuclein proteins (synucleinopathies).
E67. The method of E65, wherein the α-synuclein disease is PD.
E68. Use of one or more of the fusion protein of any one of E1-E39, a cell expressing the fusion protein of E1-E39, the nucleic acid of any one of E40-E45, the vector of any one of E46-E47, the virus particle of any one of E48-E55, and the pharmaceutical composition of E56, in preventing or delaying the progression of a α-synuclein disease in a subject.
E69. Use of one or more of the fusion protein of any one of E1-E39, a cell expressing the fusion protein of E1-E39, the nucleic acid of any one of E40-E45, the vector of any one of E46-E47, the virus particle of any one of E48-E55, and the pharmaceutical composition of E56, in the preparation of a medicament for the treatment or prevention of a α-synuclein disease in a subject.
E70. The use of E68 or E69, wherein the α-synuclein disease is PD.

### DESCRIPTION OF THE FIGURES

FIG. 1A shows a Clustal Omega sequence alignment of representative human J domain sequences. The highly conserved HPD domain is shown in the highlighted box.

FIG. 1B shows a Clustal Omega sequence alignment of representative human J domain sequences.

FIG. 2 shows some representative fusion protein constructs comprising a J domain and α-synuclein-binding domains and control constructs.

FIG. 3 shows the effect of expressing a fusion protein comprising a J domain and an α-synuclein-binding domain. FIG. 3A: Wild type (WT) α-synuclein (Syn(WT): bars 1-3) or mutant α-synuclein (Syn(A53T): bars 4-6) was expressed alone or with a Flag epitope-tagged fusion protein comprising the J-domain DnaJB1 and a α-synuclein-binding domain (J-SynBP1: bars 2 and 5) or a control fusion protein construct comprising a mutated J-domain (containing a P33Q substitution) and a α-synuclein-binding peptide (J(MT)-SynBP1: bars 3 and 6) in HEK293 cells. Two days later, cells were homogenized in Tris buffer (10 mM Tris, pH7.4, 140 mM NaCl, 1 mM EDTA supplemented with protease inhibitor cocktail). Aggregation of mutant α-synuclein levels were quantified with sandwich ELISA, in which aggregated α-synuclein proteins were captured by coated-Syn-O4 antibody, followed by detection with HRP-conjugated anti-α-synuclein antibody (BioLegend: A15115A). FIG. 3B Lysates were prepared from HEK293 cells transiently transfected with wildtype Syn(WT) (lanes 1-3) or mutant Syn(A53T) (lane 4-6) alone or co-expressed with the fusion protein constructs. After brief sonication, the cell lysate was subjected to SDS-PAGE/immuno blotting with anti-α-synuclein antibody (Ab-2: top panel), Flag antibody (middle panel) or anti-Tubulin antibody (bottom panel).

FIG. 4 shows the reduction in the amount of α-synuclein secreted into the culture medium in cells co-expressing the fusion protein comprising a J domain and α-synuclein-binding domain. Transfection of cells with wildtype (bar 2) or mutant (A53T; bar 4) α-synuclein alone results in secretion of the synucleins as detectable by ELISA. Co-expression with the fusion protein construct comprising either a normal (bar 3) or mutated J-domain (containing a P33Q substitution; bar 5) results in the elimination of detectable levels of secreted α-synuclein.

FIG. 5 shows the effect of inhibitors of various cellular processes in the total α-synuclein levels in cells expressing the fusion protein constructs. Cells were transfected with either wildtype (lane 2) or mutant (lanes 3-8) α-synuclein, and also transfected with the JB1-SynBP1 fusion protein (lanes 4-8). Cells were also treated with either Bafilomycin A1, an inhibitor of late phase autophagy (10 nM and 100 nM in lanes 5 and 6, respectively), or MG132, a proteasome inhibitor (0.1 μM and 1 μM in lanes 7 and 8, respectively). Cell lysates were probed with anti-synuclein antibodies.

FIG. 6 shows the effect of inhibitors of various cellular processes in the aggregated α-synuclein levels in cells expressing the fusion protein constructs. Cells were transfected with either wildtype or mutant α-synuclein, and also transfected with the JB1-SynBP1 fusion protein. Cells were also treated with either Bafilomycin A1, an inhibitor of late phase autophagy (10 nM and 100 nM), or MG132, a proteasome inhibitor (0.1 μM and 1 μM). Aggregated synuclein in cell lysates were determined by ELISA using an anti-aggregated synuclein antibody.

FIG. 7 shows amelioration of α-synuclein (A53T)-mediated cytotoxicity by the JB1-SynBP1 construct in U87-MG glioma cells. U87MG cells were infected with lentivirus to express either wildtype (“SynWT”) or mutant (“SynA53T”) α-synuclein protein, either alone or with the JB1-SynBP1 construct. The culture medium was collected from U87-MG cells at 7-day after infection. Lactate dehydrogenase (LDH) activity in culture medium was measured by LDH-Cytox™ Assay Kit (BioLegend), and expressed as values relative to LDH levels in cells expressing SynWT alone.

### DEFINITIONS

As used in the specification and claims, the singular forms “a”, “an” and “the” include plural references unless the context clearly dictates otherwise. For example, the term “a cell” includes a plurality of cells, including mixtures thereof.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein the term “amino acid” refers to either natural and/or unnatural or synthetic amino acids, including but not limited to both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated,” when used to describe the various polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” polynucleotide or polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The terms “α-synuclein disorder” or “synucleinopathies”, as herein defined refers to disorders associated with formation of intracellular α-synuclein aggregates, particularly aggregates of α-synuclein mutant protein. Examples of α-synuclein disorders include, but are not limited to Parkinsonism, including PD, dementia with Lewy bodies, multiple system atrophy, and diseases related to abnormal accumulation of aggregated α-synuclein proteins (synucleinopathies).

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “operably linked” refers to a juxtaposition of described components wherein the components are in a relationship permitting them to function in their intended manner. A control sequence “operably linked” to a coding sequence is ligated in such a way that expression of the coding sequence is achieved under conditions compatible with the control sequences. “Operably linked” sequences may include both expression control sequences that are contiguous with the gene of interest and expression control sequences that act in trans or at a distance to control the gene of interest. The term “expression control sequence” refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (such as, a Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing and can also include additional components whose presence is advantageous, for example, leader sequences and fusion partner sequences. Unless stated otherwise, a description or statement herein of inserting a nucleic acid molecule encoding a fusion protein of the invention into an expression vector means that the inserted nucleic acid has also been operably linked within the vector to a functional promoter and other transcriptional and translational control elements required for expression of the encoded fusion protein when the expression vector containing the inserted nucleic acid molecule is introduced into compatible host cells or compatible cells of an organism.

“Recombinant” as applied to a polynucleotide means that the polynucleotide is the product of various combinations of in vitro cloning, restriction and/or ligation steps, and other procedures that result in a construct that can potentially be expressed in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

The terms “disease” and “disorder” are used interchangeably to indicate a pathological state identified according to acceptable medical standards and practices in the art.

As used herein, the term “effective amount” refers to the amount of a therapy that is sufficient to reduce or ameliorate the severity and/or duration of a disease or one or more symptoms thereof; to prevent the advancement of a detrimental or pathological state; to cause regression of a pathological state; to prevent recurrence, development, onset, or progression of one or more symptoms associated with a pathological state; to detect a disorder; or to enhance or improve the prophylactic or therapeutic effect(s) of a therapy (e.g., the administration of another prophylactic or therapeutic agent).

As used herein, the term “J domain” refers to a fragment which retains the ability to accelerate the intrinsic ATPase catalytic activity of Hsp70 and its cognate. The J domains of a variety of J proteins have been determined (see, for example, Kampinga et al. (2010) Nat. Rev., 11: 579-592; Hennessy et al. (2005) Protein Science, 14: 1697-1709, each of which is incorporated by reference in its entirety), and are characterized by a number of hallmarks: which is characterized by four α-helices (I, II, III, IV) and usually having the highly conserved tripeptide sequence motif of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is necessary for stimulation of Hsp70 ATPase activity. As used herein, the term “J domain” is meant to include natural J domain sequences and functional variants thereof which retain the ability to accelerate Hsp70 intrinsic ATPase activity, which can be measured using methods well known in the art (see, for example, Horne et al. (2010) J. Biol. Chem., 285, 21679-21688, which is incorporated herein by reference in its entirety). A non-limiting list of human J domains is provided in Table 1.

### DETAILED DESCRIPTION

The present inventors have found that certain contacting cells with a fusion protein construct comprising a J domain of a J protein and a α-synuclein-binding domain have the unexpected effect of reducing the aggregation of α-synuclein proteins. Aggregation of mutant α-synuclein are believed to cause a number of devastating diseases, including, but not limited to, Parkinsonism, Lewy Body dementia, multiple system atrophy, diseases related to abnormal accumulation of aggregated α-synuclein proteins (synucleinopathies). Accordingly, useful compositions and methods to treat α-synuclein disorders, e.g., in a subject in need thereof, are provided herein.

To overcome issues associated with chaperone-based therapies, we investigated whether it would be possible to design artificial chaperone proteins with high specificity. We designed a series of fusion protein constructs comprising an effector domain for Hsp70 binding/activation (J domain sequence), and a domain conferring specificity to α-synuclein proteins. The resulting fusion proteins act to accelerate the intrinsic ATPase catalytic activity of Hsp70 and its cognate, resulting in increased protein folding, reduced aggregation and/or accelerated clearance.

I. Fusion Protein Constructs

a. J Domains Useful in the Invention

J domains of a variety of J proteins have been determined. See, for example, Kampinga et al., Nat. Rev., 11: 579-592 (2010); Hennessy et al., Protein Science, 14:1697-1709 (2005). A J domain useful in preparing a fusion protein of the invention has the key defining features of a J domain which principally accelerates HSP70 ATPase activity. Accordingly, an isolated J domain useful in the invention comprises a polypeptide domain, which is characterized by four α-helices (I, II, III, IV) and usually having the highly conserved tripeptide sequence of histidine, proline, and aspartic acid (referred to as the “HPD motif”) between helices II and III. Typically, the J domain of a J protein is between fifty and seventy amino acids in length, and the site of interaction (binding) of a J domain with an Hsp70-ATP chaperone protein is believed to be a region extending from within helix II and the HPD motif is fundamental to primitive activity. Representative J domains include, but are not limited, a J domain of a DnaJB1, DnaJB2, DnaJB6, DnaJC6, a J domain of a large T antigen of SV40, and a J domain of a mammalian cysteine string protein (CSP-α). The amino acid sequences for these and other J domains that may be used in fusion proteins of the invention are provided in Table 1. The conserved HPD motif is highlighted in bold.

TABLE 1
Representative Human J Domain Sequences

| Protein | SEQ ID | | |
|---|---|---|---|
| Name | NO: | Length | J domain amino acid sequence |
| DNAJA1 | 1 | 63 | TYYDVLGVKPNATQEELKKAYRKLALKYHPDKNPNEGEKF |
| | | | KQISQAYEVLSDAKKRELYDKGG |
| DNAJA2 | 2 | 63 | KLYDILGVPPGASENELKKAYRKLAKEYHPDKNPNAGDKF |
| | | | KEISFAYEVLSNPEKRELYDRYG |
| DNAJA3 | 3 | 66 | DYYQILGVPRNASQKEIKKAYYQLAKKYHPDTNKDDPKAK |
| | | | EKFSQLAEAYEVLSDEVKRKQYDAYG |
| DNAJA4 | 4 | 67 | ETQYYDILGVKPSASPEEIKKAYRKLALKYHPDKNPDEGE |
| | | | KFKLISQAYEVLSDPKKRDVYDQGGEQ |
| DNAJB1 | 5 | 69 | GKDYYQTLGLARGASDEEIKRAYRRQALRYHPDKNKEPGA |
| | | | EEKFKEIAEAYDVLSDPRKREIFDRYGEE |
| DNAJB2 | 6 | 70 | ASYYEILDVPRSASADDIKKAYRRKALQWHPDKNPDNKEF |
| | | | AEKKFKEVAEAYEVLSDKHKREIYDRYGRE |
| DNAJB3 | 7 | 69 | MVDYYEVLDVPRQASSEAIKKAYRKLALKWHPDKNPENKE |
| | | | EAERRFKQVAEAYEVLSDAKKRDIYDRYG |
| DNAJB4 | 8 | 69 | GKDYYCILGIEKGASDEDIKKAYRKQALKFHPDKNKSPQA |
| | | | EEKFKEVAEAYEVLSDPKKREIYDQFGEE |
| DNAJB5 | 9 | 65 | DYYKILGIPSGANEDEIKKAYRKMALKYHPDKNKEPNAEE |
| | | | KFKEIAEAYDVLSDPKKRGLYDQYG |
| DNAJB6 | 10 | 68 | VDYYEVLGVQRHASPEDIKKAYRKLALKWHPDKNPENKEE |
| | | | AERKFKQVAEAYEVLSDAKKRDIYDKYG |
| DNAJB7 | 11 | 67 | DYYEVLGLQRYASPEDIKKAYHKVALKWHPDKNPENKEEA |
| | | | ERKFKEVAEAYEVLSNDEKRDIYDKYG |
| DNAJB8 | 12 | 67 | NYYEVLGVQASASPEDIKKAYRKLALRWHPDKNPDNKEEA |
| | | | EKKFKLVSEAYEVLSDSKKRSLYDRAG |
| DNAJB9 | 13 | 65 | SYYDILGVPKSASERQIKKAFHKLAMKYHPDKNKSPDAEA |
| | | | KFREIAEAYETLSDANRRKEYDTLG |
| DNAJB11 | 14 | 66 | DFYKILGVPRSASIKDIKKAYRKLALQLHPDRNPDDPQAQ |
| | | | EKFQDLGAAYEVLSDSEKRKQYDTYG |
| DNAJB12 | 15 | 65 | YEILGVSRGASDEDLKKAYRRLALKFHPDKNHAPGATEAF |
| | | | KAIGTAYAVLSNPEKRKQYDQFGDD |
| DNAJB13 | 16 | 65 | DYYSVLGITRNSEDAQIKQAYRRLALKHHPLKSNEPSSAE |
| | | | IFRQIAEAYDVLSDPMKRGIYDKFG |
| DNAJB14 | 17 | 65 | NYYEVLGVTKDAGDEDLKKAYRKLALKFHPDKNHAPGATD |
| | | | AFKKIGNAYAVLSNPEKRKQYDLTG |
| DNAJC1 | 18 | 65 | NFYQFLGVQQDASSADIRKAYRKLSLTLHPDKNKDENAET |
| | | | QFRQLVAIYEVLKDDERRQRYDDIL |
| DNAJC2 | 19 | 74 | DHYAVLGLGHVRYKATQRQIKAAHKAMVLKHHPDKRKAAG |
| | | | EPIKEGDNDYFTCITKAYEMLSDPVKRRAFNSVD |
| DNAJC3 | 20 | 69 | DYYKILGVKRNAKKQEIIKAYRKLALQWHPDNFQNEEEKK |
| | | | KAEKKFIDIAAAKEVLSDPEMRKKFDDGE |
| DNAJC4 | 21 | 66 | TYYELLGVHPGASTEEVKRAFFSKSKELHPDRDPGNPSLH |
| | | | SRFVELSEAYRVLSREQSRRSYDDQL |
| DNAJC5 | 22 | 70 | GESLYHVLGLDKNATSDDIKKSYRKLALKYHPDKNPDNPE |
| | | | AADKFKEINNAHAILTDATKRNIYDKYGSL |
| DNAJC5B | 23 | 66 | ALYEILGLHKGASNEEIKKTYRKLALKHHPDKNPDDPAAT |
| | | | EKFKEINNAHAILTDISKRSIYDKYG |
| DNAJC6 | 24 | 65 | TKWKPVGMADLVTPEQVKKVYRKAVLVVHPDKATGQPYEQ |
| | | | YAKMIFMELNDAWSEFENQGQKPLY |
| DNAJC7 | 25 | 71 | DYYKILGVDKNASEDEIKKAYRKRALMHHPDRHSGASAEV |
| | | | QKEEEKKFKEVGEAFTILSDPKKKTRYDSGQ |
| DNAJC8 | 26 | 68 | NPFEVLQIDPEVTDEEIKKRFRQLSILVHPDKNQDDADRA |
| | | | QKAFEAVDKAYKLLLDQEQKKRALDVIQ |
| DNAJC9 | 27 | 68 | DLYRVLGVRREASDGEVRRGYHKVSLQVHPDRVGEGDKED |
| | | | ATRRFQILGKVYSVLSDREQRAVYDEQG |
| DNAJC10 | 28 | 66 | DFYSLLGVSKTASSREIRQAFKKLALKLHPDKNPNNPNAH |
| | | | GDFLKINRAYEVLKDEDLRKKYDKYG |
| DNAJC11 | 29 | 69 | DYYSLLNVRREASSEELKAAYRRLCMLYHPDKHRDPELKS |
| | | | QAERLFNLVHQAYEVLSDPQTRAIYDIYG |
| DNAJC12 | 30 | 66 | DYYTLLGCDELSSVEQILAEFKVRALECHPDKHPENPKAV |
| | | | ETFQKLQKAKEILTNEESRARYDHWR |
| DNAJC13 | 31 | 66 | DAYEVLNLPQGQGPHDESKIRKAYFRLAQKYHPDKNPEGR |
| | | | DMFEKVNKAYEFLCTKSAKIVDGPDP |
| DNAJC14 | 32 | 65 | NPFHVLGVEATASDVELKKAYRQLAVMVHPDKNHHPRAEE |
| | | | AFKVLRAAWDIVSNAEKRKEYEMKR |
| DNAJC15 | 33 | 55 | EAGLILGVSPSAGKAKIRTAHRRVMILNHPDKGGSPYVAA |
| | | | KINEAKDLLETTTKH |
| DNAJC16 | 34 | 65 | DPYRVLGVSRTASQADIKKAYKKLAREWHPDKNKDPGAED |
| | | | KFIQISKAYEILSNEEKRSNYDQYG |
| DNAJC17 | 35 | 66 | DLYALLGIEEKAADKEVKKAYRQKALSCHPDKNPDNPRAA |
| | | | ELFHQLSQALEVLTDAAARAAYDKVR |
| DNAJC18 | 36 | 65 | NYYEILGVSRDASDEELKKAYRKLALKFHPDKNCAPGATD |
| | | | AFKAIGNAFAVLSNPDKRLRYDEYG |
| DNAJC19 | 37 | 55 | EAALILGVSPTANKGKIRDAHRRIMLLNHPDKGGSPYIAA |
| | | | KINEAKDLLEGQAKK |
| DNAJC20 | 38 | 72 | DYFSLMDCNRSFRVDTAKLQHRYQQLQRLVHPDFFSQRSQ |
| | | | TEKDFSEKHSTLVNDAYKTLLAPLSRGLYLLK |
| DNAJC21 | 39 | 67 | CHYEALGVRRDASEEELKKAYRKLALKWHPDKNLDNAAEA |
| | | | AEQFKLIQAAYDVLSDPQERAWYDNHR |
| DNAJC22 | 40 | 65 | LAYQVLGLSEGATNEEIHRSYQELVKVWHPDHNLDQTEEA |
| | | | QRHFLEIQAAYEVLSQPRKPWGSRR |
| DNAJC23 | 41 | 62 | NPYEVLNLDPGATVAEIKKQYRLLSLKYHPDKGGDEVMFM |
| | | | RIAKAYAALTDEESRKNWEEFG |
| DNAJC24 | 42 | 72 | DWYSILGADPSANISDLKQKYQKLILMYHPDKQSTDVPAG |
| | | | TVEECVQKFIEIDQAWKILGNEETKREYDLQR |
| DNAJC25 | 43 | 76 | DCYEVLGVSRSAGKAEIARAYRQLARRYHPDRYRPQPGDE |
| | | | GPGRTPQSAEEAFLLVATAYETLKDEETRKDYDYML |
| DNAJC26 | 44 | 65 | SRWTPVGMADLVAPEQVKKHYRRAVLAVHPDKAAGQPYEQ |
| | | | HAKMIFMELNDAWSEFENQGSRPLF |
| DNAJC27 | 45 | 57 | DSWDMLGVKPGASRDEVNKAYRKLAVLLHPDKCVAPGSED |
| | | | AFKAWNARTALLKNIK |
| DNAJC28 | 46 | 65 | EYYRLLNVEEGCSADEVRESFHKLAKQYHPDSGSNTADSA |
| | | | TFIRIEKAYRKVLSHVIEQTNASQS |
| DNAJC29 | 47 | 88 | ILKEVTSVVEQAWKLPESERKKIIRRLYLKWHPDKNPENH |
| | | | DIANEVFKHLQNEINRLEKQAFLDQNADRASRRTFSTSAS |
| | | | RFQSDKYS |
| DNAJC30 | 48 | 66 | ALYDLLGVPSTATQAQIKAAYYRQCFLYHPDRNSGSAEAA |
| | | | ERFTRISQAYVVLGSATLRRKYDRGL |
| SV40 | 49 | 64 | QLMDLLGLERSAWGNIPLMRKAYLKKCKEFHPDKGGDEEK |
| Jdomain | | | MKKMNTLYKKMEDGVKYAHQPDFG |
| Bacterial | 50 | 70 | KQDYYEILGVSKTAEEREIRKAYKRLAMKYHPDRNQGDKE |
| J-domain | | | AEAKFKEIKEAYEVLTDSQKRAAYDQYGHA |

In one embodiment, the fusion protein comprises a J domain sequence selected from the polypeptide sequences selected from the group consisting of SEQ ID NOs: 1-50. The inventors have demonstrated that J domains comprising the conserved “HPD” motif have activity (data not shown). Therefore, in another embodiment, the fusion protein comprises a J domain sequence comprising the conserved “HPD” motif. For example, in a particular embodiment, the fusion protein comprises a J domain sequence selected from the polypeptide sequences selected from the group consisting of SEQ ID NOs: 1-15 and 17-50. In another embodiment, the fusion protein comprises a J domain sequence selected from the polypeptide sequences selected from the group consisting of SEQ ID NOs: 1, 5, 6, 10, 16, 24, 25, 31 and 49.

b. α-Synuclein-Binding Domain

The fusion protein also comprises at least one α-synuclein-binding domain. The α-synuclein-binding domain can be a single chain polypeptide, or a multimeric polypeptide joined with the J domain to form the fusion protein.

It is ideal that the α-synuclein-binding domain possesses a sufficient affinity to be able to bind the α-synuclein protein when present at a pathological level within cells. Therefore, in one embodiment, the fusion protein comprises a α-synuclein-binding domain that has a K_{D} for α-synuclein of, for example, 2 μM or less, 1 μM or less, 500 nM or less, 300 nM or less, 100 nM or less, 30 nM or less when tested by ELISA on 96 well microtiter plates. In another embodiment, the fusion protein comprises a α-synuclein-binding domain that has a K_{D} for the aggregated form of α-synuclein of, for example, 2 μM or less, 1 μM or less, 500 nM or less, 300 nM or less, 100 nM or less, 30 nM or less when tested by ELISA on 96 well microtiter plates. In still another embodiment, the α-synuclein-binding domain has selectivity for the aggregated form of α-synuclein; for example, the α-synuclein-binding domain has at least two-fold higher, e.g., at least 3 fold higher, at least 4 fold higher, at least 5 fold higher, at least 10 fold higher, at least 30 fold higher, at least 100 fold higher affinity for the aggregated form of α-synuclein when compared with the affinity for the soluble form of α-synuclein.

α-synuclein-binding domains have been previously identified and characterized (see, for example, U.S. Pat. No. 7,605,133, WO 2019/161386, Abe et al., (2007) BMC Bioinformatics, 8:451, each of which is incorporated herein by reference). Therefore, in another embodiment, the fusion protein comprises a α-synuclein-binding that is selected from the group consisting of SEQ ID NOs: 51-64 (see, for example, Table 2). In one particular embodiment, the fusion protein comprises the α-synuclein-binding domain of SEQ ID NO: 51. In another embodiment, the fusion protein comprises the α-synuclein-binding domain of SEQ ID NO: 63.

In still another embodiment, the fusion protein comprises a J domain and a α-synuclein-binding domain, wherein the α-synuclein-binding domain does not comprise the sequence of SEQ ID NO:65. In still another embodiment, the fusion protein comprises a J domain and a α-synuclein-binding domain, wherein the fusion protein does not comprise the sequence of SEQ ID NO:65. In a particular embodiment, the fusion protein comprises a J domain and a α-synuclein-binding domain, wherein the fusion protein does not comprise the sequence of SEQ ID NO:65, and the J domain is selected from the group consisting of SEQ ID NOs: 1, 5, 6, 10, 16, 24, 25, 31 and 49, and the α-synuclein-binding domain is attached to the C-terminus of the J domain, with or without an optional linker.

In another aspect, the fusion protein comprises a J domain and the target binding protein SEQ ID NO: 65. Fusion protein constructs comprising QBP1 (SEQ ID NO: 65) was previously shown to reduce the formation of mechanostable and hyper-mechanostable conformers in A53T α-synuclein (Hervás et al., (2012) PLoS Biol. 10(5):e1001335). Therefore, in a particular embodiment, the fusion protein comprises the α-synuclein-binding domain of SEQ ID NO: 65. In a particular embodiment, the fusion protein construct comprises a J domain sequence selected from the group consisting of SEQ ID NOs: 1, 5, 6, 10, 16, 24, 25, 31 and 49, and the α-synuclein-binding domain of SEQ ID NO: 65. In one embodiment, the fusion protein construct comprises the J domain sequence of SEQ ID NO: 5 and the α-synuclein-binding domain of SEQ ID NO: 65.

In another embodiment, the fusion protein also contemplates the use of the α-synuclein-binding domain that is chemically conjugated to the J domain. The α-synuclein-binding domain can be conjugated directly to the J domain. Alternatively, it can be conjugated to the J domain by a linker. For example, there are a large number of chemical cross-linking agents that are known to those skilled in the art and useful for cross-linking the α-synuclein-binding domain to the J domain, or a targeting domain to a fusion protein comprising the α-synuclein-binding domain and J domain. For example, the cross-linking agents are heterobifunctional cross-linkers, which can be used to link molecules in a stepwise manner. Heterobifunctional cross-linkers provide the ability to design more specific coupling methods for conjugating proteins, thereby reducing the occurrences of unwanted side reactions such as homo-protein polymers. A wide variety of heterobifunctional cross-linkers are known in the art, including succinimidyl 4-(N-maleimidomethyl)cyclohexane-1-carboxylate (SMCC), m-Maleimidobenzoyl-N-hydroxysuccinimide ester (MBS); N-succinimidyl(4-iodoacetyl)aminobenzoate (SIAB), succinimidyl 4-(p-maleimidophenyl)butyrate (SMPB), 1-ethyl-3-(3-dimethylaminopropyl)carbodiimide hydrochloride (EDC); 4-succinimidyloxycarbonyl-a-methyl-a-(2-pyridyldithio)-toluene (SMPT), N-succinimidyl 3-(2-pyridyldithio)propionate (SPDP), succinimidyl 6-[3-(2-pyridyldithio)propionate]hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo. In addition to the heterobifunctional cross-linkers, there exists a number of other cross-linking agents including homobifunctional and photoreactive cross-linkers. Disuccinimidyl suberate (DSS), bismaleimidohexane (BMH) and dimethylpimelimidate.2 HCl (Forbes-Cori Disease) are examples of useful homobifunctional cross-linking agents, and bis-[B-(4-azidosalicylamido)ethyl]disulfide (BASED) and N-succinimidyl-6(4′-azido-2′-nitrophenylamino)hexanoate (SANPAH) are examples of useful photoreactive cross-linkers for use in this disclosure. For a recent review of protein coupling techniques, see Means et al., (1990) Bioconj. Chem. 1:2-12, incorporated by reference herein.

TABLE 2
Examples of α-synuclein-binding domains

| | SEQ ID | | |
|---|---|---|---|
| Name | NO: | Length | Sequence |
| SynBP1 | 51 | 10 | KDGIVNGVKA |
| SynBP2 | 52 | 7 | WRQTRKD |
| SynBP3 | 53 | 7 | HYAKNPI |
| SynBP4 | 54 | 7 | ATINKSL |
| SynBP5 | 55 | 7 | RRRGMAI |
| SynBP6 | 56 | 7 | TKHGPRK |
| SynBP7 | 57 | 7 | SLKRLPK |
| SynBP8 | 58 | 7 | RLRGRNQ |
| SynBP9 | 59 | 7 | WPFHHHR |
| SynBP10 | 60 | 7 | HLYHHKT |
| SynBP11 | 61 | 7 | THIHHPS |
| SynBP12 | 62 | 7 | MMMMMRL |
| NAC32 | 63 | 248 | QVQLQQSGPGLVKPSQTLSLTCAISGDSVSSNSAAWNWI |
| (intra body) | | | RQSPSRGLEWLGRTFYRSKWYNDYAASVKSRITIDPDTS |
| | | | KNQFSLQLNSVTPEDTAVYYCTRQNLAGPFDSWGQGTLV |
| | | | TVSSGILGSGGGGSGGGGSGGGGSEIVMTQSPGTLSLSP |
| | | | GERATLSCRASQSVSSNYLAWYQQKAGQAPRLLISGASS |
| | | | RATGIPDRFSGSGSGTDFTLTISRLEPEDFAVYYCQQYG |
| | | | SSTAFGPGTKVDIK |
| Syn_scFv | 64 | 247 | LQSVLTQPPSVSGAPGQRVAISCTGTSSDIGTGYDVNWY |
| | | | QHLPGTAPRLLIYGNTYRPSGVPDRFSASTGSKSGTSAS |
| | | | LVITDLQAEDEGDYYCQSFDNSLRGSVFGGGTKVTVLGE |
| | | | GKSSGSGSESKASEVQLVQSGGGVVKPGGSLRLSCEASG |
| | | | FILSDYYMTWIRQAPGKGLEWLAVIDITSSYTNYADSVK |
| | | | GRFTISRDNAKNSVYLQMNSLRAEDTAVYYCARLESGFF |
| | | | DYWGQGTLVTVSS |

c. Optional Linker

The fusion proteins described herein can optionally contain one or more linkers. Linkers can be peptidic or non-peptidic. The purpose of the linker is to provide, among other things, an adequate distance between functional domains within the protein (e.g., between the J domain and α-synuclein-binding domain, between tandem arrangements of α-synuclein-binding domains, between either the J domain and α-synuclein-binding domain and an optional targeting reagent, or between either the J domain and α-synuclein-binding domain and an optional detection domain or epitope) for optimal function of each of the domains. Clearly, a linker preferably does not interfere with the respective functions of the J domain, the target protein binding domain of a fusion protein according to the invention. A linker, if present in a fusion protein of the invention, is selected to attenuate the cytotoxicity caused by target proteins (α-synuclein proteins), and it may be omitted if direct attachment achieves a desired effect. Linkers present in a fusion protein of the invention may comprise one or more amino acids encoded by a nucleotide sequence present on a segment of nucleic acid in or around a cloning site of an expression vector into which is inserted in frame a nucleic acid segment encoding a protein domain or an entire fusion protein as described herein. In one embodiment, the peptide linker is between 1 amino acid and 20 amino acids in length. In another embodiment, the peptide linker is between 2 amino acids and 15 amino acids in length. In still another embodiment, the peptide linker is between 2 amino acids and 10 amino acids in length.

Selecting one or more polypeptide linkers to produce a fusion protein according to the invention is within the knowledge and skill of practitioners in the art. See, for example, Arai et al., Protein Eng., 14(8): 529-532 (2001); Crasto et al., Protein Eng., 13(5): 309-314 (2000); George et al., Protein Eng., 15(11): 871-879 (2003); Robinson et al., Proc. Natl. Acad. Sci. USA, 95: 5929-5934 (1998), each of which is incorporated herein by reference in its entirety. Examples of linkers of two or more amino acids that may be used in preparing a fusion protein according to the invention, include, by are not limited to, those provided below in Table 3.

TABLE 3
Linker Sequences

| SEQ ID NO: | Length | Sequence |
|---|---|---|
| 66 | 2 | SR |
| 67 | 4 | GTGS |
| 68 | 5 | GLESR |
| 69 | 4 | GGSG |
| 70 | 5 | GGGGS |
| 71 | 5 | DIAAA |
| 72 | 5 | EAAAK |
| 73 | 15 | GGGGSGGGGSGGGGS |
| 74 | 11 | AEAAAKEAAAK |
| 75 | 15 | SGGGSGGGGSGGGGS |
| 76 | 25 | DIGGGGSGGGGSGGGGSGGGGSAAA |

d. Targeting Reagents

The fusion proteins disclosed herein can further comprise a targeting moiety. As used herein, the terms “targeting moiety” and “targeting reagent” are used interchangeably and refer to a substance associated with the fusion protein that enhances binding, transport, accumulation, residence time, bioavailability, or modifies biological activity or therapeutic effect of the fusion protein in a cell or in the body of a subject. A targeting moiety can have functionality at the tissue, cellular, and/or subcellular level. The targeting moiety can direct localization of the fusion protein to a particular cell, tissue or organ, or intracellular distribution, for example, upon administration of the fusion protein into a subject. In one embodiment, the targeting moiety is located at the N-terminus of the fusion protein. In another embodiment, the targeting moiety is located at the C-terminus of the fusion protein. In still another embodiment, the targeting moiety is located internally. In another embodiment, the targeting moiety is attached to the fusion protein via chemical conjugation. In further embodiment, the targeting moiety can be an amino acid sequence for subcellular localization such as nuclear localization signal or nuclear export signal. The targeting moiety can include, but is not limited to, an organic or inorganic molecule, a peptide, a peptide mimetic, a protein, an antibody or fragment thereof, a growth factor, an enzyme, a lectin, an antigen or immunogen, viruses or component thereof, a viral vector, a receptors, a receptor ligand, a toxins, a polynucleotide, an oligonucleotide or aptamer, a nucleotide, a carbohydrate, a sugar, a lipid, a glycolipid, a nucleoprotein, a glycoprotein, a lipoprotein, a steroid, a hormone, a growth factor, a chemoattractant, a cytokine, a chemokine, a drug, or a small molecule, among others.

In an exemplary embodiment of the present invention, the targeting moiety enhances binding, transport, accumulation, residence time, bioavailability, or modifies biological activity of the modifies biological activity or therapeutic effect of the platform, or its associated ligand and/or active agent in the target cell or tissue, for example, neuronal cells, the central nervous system, and/or the peripheral nervous system. Thus, the targeting moiety can have specificity for cellular receptors associated with the central nervous system, or is otherwise associated with enhanced delivery to the CNS via the blood-brain barrier (BBB). Consequently, a ligand, as described above, can be both a ligand and a targeting moiety.

In some embodiments, the targeting moiety can be a cell-penetrating peptide, for example, as described in U.S. Pat. No. 10,111,965, which is incorporated by reference in its entirety. In another embodiment, the targeting moiety can be an antibody or an antigen-binding fragment or single-chain derivative thereof, for example, as described in U.S. Ser. No. 16/131,591, which is incorporated herein by reference in its entirety. The targeting moiety can be coupled to the platform for targeted cellular delivery by being directly or indirectly bound to the core. For example, in embodiments where the core comprises a nanoparticle, conjugation of the targeting moiety to the nanoparticle can utilize similar functional groups that are employed to tether PEG to the nanoparticle. Thus, the targeting moiety can be directly bound to the nanoparticle through functionalization of the targeting moiety. Alternatively, the targeting moiety can be indirectly bound to the nanoparticle through conjugation of the targeting moiety to a functionalized PEG, as discussed above. A targeting moiety can be attached to core by way of covalent, non-covalent, or electrostatic interactions. In one embodiment, the targeting moiety is a peptide. In a particular embodiment, the targeting moiety is a peptide that is covalently attached to the N-terminus of the fusion protein.

e. Epitopes

In certain embodiments, the fusion protein of the present invention contains an optional epitope or tag, which can impart additional properties to the fusion protein. As used herein, the terms “epitope” and “tag” are used interchangeably to refer to an amino acid sequence, typically 300 amino acids or less in length, which is typically attached to the N-terminal or C-terminal end of the fusion protein. In one embodiment, the fusion protein of the present invention further comprises an epitope which is used to facilitate purification. Examples of such epitopes useful for purification, provided below in Table 4, include the human IgG1 Fc sequence (SEQ ID NO: 77), the FLAG epitope (DYKDDDDK, SEQ ID NO: 78), His6 epitope (SEQ ID NO: 79), c-myc (SEQ ID NO: 80), HA (SEQ ID NO: 81), V5 epitope (SEQ ID NO: 82), or glutathione-s-transferase (SEQ ID NO: 83). In another embodiment, the fusion protein of the present invention further comprises an epitope which is used to increase the half-life of the fusion protein when administered into a subject, for example a human. Examples of such epitopes useful for increasing half-life include the human Fc sequence. Therefore, in one particular embodiment, the fusion protein comprises, in addition to a J domain and α-synuclein-binding domain, a human Fc epitope. The epitope is positioned at the C-terminal end of the fusion protein.

TABLE 4
Representative Examples of Epitopes

| SEQ ID NO: | EPITOPE | LENGTH | SEQUENCE |
|---|---|---|---|
| 77 | Human IgG1 | 232 | EPKSCDKTHTCPPCPAPELLGGPSVFLFPPKPKDTLMISRTPE |
| | Fc domain | | VTCVVVDVSHEDPEVKFNWYVDGVEVHNAKTKPREEQYN |
| | | | STYRVVSVLTVLHQDWLNGKEYKCKVSNKALPAPIEKTISKA |
| | | | KGQPREPQVYTLPPSRDELTKNQVSLTCLVKGFYPSDIAVEW |
| | | | ESNGQPENNYKTTPPVLDSDGSFFLYSKLTVDKSRWQQGN |
| | | | VFSCSVMHEALHNHYTQKSLSLSPGK |
| 78 | FLAG epitope | 8 | DYKDDDDK |
| 79 | His6 | 6 | HHHHHH |
| 80 | c-myc | 10 | EQKLISEEDL |
| 81 | HA | 9 | YPYDVPDYA |
| 82 | V5 epitope | 14 | GKPIPNPLLGLDST |
| 83 | Glutathione- | 220 | MSPILGYWKIKGLVQPTRLLLEYLEEKYEEHLYERDEGDKWR |
| | S-transferase | | NKKFELGLEFPNLPYYIDGDVKLTQSMAIIRYIADKHNMLGG |
| | | | CPKERAEISMLEGAVLDIRYGVSRIAYSKDFETLKVDFLSKLPE |
| | | | MLKMFEDRLCHKTYLNGDHVTHPDFMLYDALDVVLYMDP |
| | | | MCLDAFPKLVCFKKRIEAIPQIDKYLKSSKYIAWPLQGWQAT |
| | | | FGGGDHPPKSD |

f. Cell-Penetrating Peptides

In still other embodiments, the fusion protein described herein can further comprise a cell-penetrating peptide. Cell-penetrating peptides are known to carry a conjugated cargo, whether a small molecule, peptide, protein or nucleic acid, into cells. Non-limiting examples of cell-penetrating peptides in a fusion protein of the invention include, but are not limited to, a polycationic peptide, e.g., an HIV TAT peptide49-57, polyarginines, and penetratin pAntan(43-58), amphipathic peptide, e.g., pep-1, a hydrophobic peptide, e.g., a C405Y, and the like. See Table 5 below.

TABLE 5
Examples of Cell-Penetrating Peptides

| SEQ ID NO: | Length | SEQUENCE |
|---|---|---|
| 84 | 9 | RKKRRQRRR |
| 85 | 15 | RQIKWFQNRRMKWKK |
| 86 | 21 | KETWWETWWTEWSQPKKKRKV |
| 87 | 17 | CSIPPEVKFNKPFVYLI |

Therefore, in one embodiment, the fusion protein comprises a cell-penetrating peptide and a fusion protein, wherein the cell-penetrating peptide is selected from the group consisting of SEQ ID NOs: 84-87, and the fusion protein is selected from the group consisting of SEQ ID NOs: 88, 90-96, 98-100. In another embodiment, the fusion protein comprises the cell-penetrating peptide of SEQ ID NO: 84, and the fusion protein selected from the group consisting of SEQ ID NOs: 88, 90-96, 98-100. In another embodiment, the fusion protein comprises the cell-penetrating peptide of SEQ ID NO: 85, and the fusion protein selected from the group consisting of SEQ ID NOs: 88, 90-96, 98-100. In still another embodiment, the fusion protein comprises the cell-penetrating peptide of SEQ ID NO: 86, and the fusion protein selected from the group consisting of SEQ ID NOs: 88, 90-96, 98-100. In yet another embodiment, the fusion protein comprises the cell-penetrating peptide of SEQ ID NO: 87, and the fusion protein selected from the group consisting of SEQ ID NOs: 88, 90-96, 98-100. Cells expressing the fusion protein constructs with the cell-penetrating peptide can be administered to a subject, for example a human subject (e.g., a patient having or at risk of suffering from a α-synuclein disorder). The fusion protein is secreted from the cells, which help reduce α-synuclein-containing protein aggregation and/or associated cytotoxicity.

g. Arrangement of J Domain and α-Synuclein Binding Domain

The fusion proteins described herein can be arranged in a multitude of ways. In one embodiment, the α-synuclein-binding domains attached to the C-terminal side of the J domain. In another embodiment, the α-synuclein-binding domains attached to the N-terminal side of the J domain. The α-synuclein-binding domain and the J domain, in either configuration, can optionally be separated via a linker as described above.

In some embodiments, the J domain can be attached to a plurality of α-synuclein-binding domains, for example, two α-synuclein-binding domains, three α-synuclein-binding domains, four α-synuclein-binding domains or more. The α-synuclein-binding domains can be attached to the N-terminal side of the J domain. Alternatively, the α-synuclein-binding domains can be attached to the C-terminal side of the J domain. In still another embodiment, the α-synuclein-binding domains can be attached on the N-terminal and C-terminal sides of the J domain. Each of the plurality of α-synuclein-binding domains can be the same α-synuclein-binding domain. In another embodiment, each of the plurality of α-synuclein-binding domains in the fusion protein can be different α-synuclein-binding domains (i.e., different sequences).

In some embodiments, the fusion proteins can comprise a structure selected from the following group:

| | |
|---|---|
| a. | DNAJ-X-S, |
| b. | DNAJ-X-S-X-S, |
| C. | DNAJ-X-S-X-S-X-S, |
| d. | S-X-DNAJ, |
| e. | S-X-S-X-DNAJ, |
| f. | S-X-S-X-S-X-DNAJ, |
| g. | S-X-DNAJ-X-S, |
| h. | S-X-DNAJ-X-S-X-S, |
| i. | S-X-S-X-DNAJ-X-S-X-S-X-S, |
| j. | S-X-S-X-S-X-DNAJ-X-S, |
| k. | S-X-S-X-S-X-DNAJ-X-S-X-S, |
| l. | S-X-S-X-S-X-DNAJ-X-S-X-S-X-S, |
| m. | DnaJ-X-DnaJ-X-X-X-S, |
| n. | S-X-DnaJ-X-DnaJ, |
| | and |
| o. | S-X-S-X-DnaJ-X-DnaJ, |

wherein,
S is a α-synuclein-binding domain,
DNAJ is a J domain of a J protein, and
X is an optional linker.

In one embodiment, the fusion protein comprises the J domain selected from the group consisting of SEQ ID NOs: 1-15 and 17-50. In another embodiment, the fusion protein comprises the J domain selected from the group consisting of SEQ ID NOs: 1, 5, 6, 10, 16, 24, 25, 31 and 49. In one particular embodiment, the fusion protein comprises the J domain of SEQ ID NO: 5.

In another embodiment, the α-synuclein-binding domain is selected from the group consisting of SEQ ID NOs: 51-64. In one particular embodiment, the α-synuclein-binding domain is SEQ ID NO:49. In another embodiment, the α-synuclein-binding domain is SEQ ID NO:63. In still another embodiment, the α-synuclein-binding domain is SEQ ID NO:64.

In still another embodiment, the fusion protein comprises the J domain of SEQ ID NO: 5, and the α-synuclein-binding domain of SEQ ID NO: 51. In another embodiment, the fusion protein comprises the J domain of SEQ ID NO: 5, and at least two copies of the α-synuclein-binding domain of SEQ ID NO: 51.

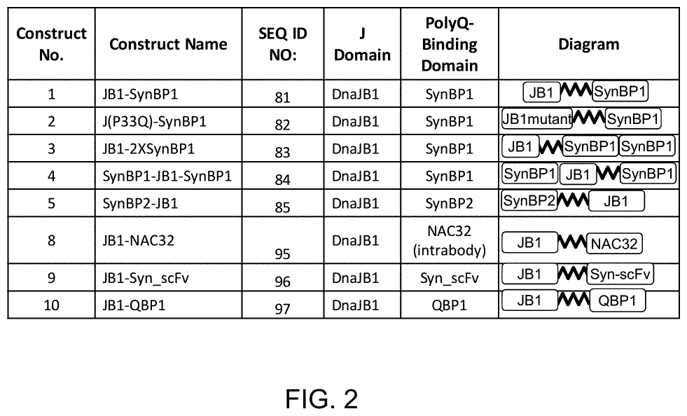

Non-limiting examples of fusion protein constructs comprising a J domain and α-synuclein-binding domain, as well as control constructs, are depicted schematically in FIG. 2, and also shown below in Table 6. In one embodiment, the specific fusion protein construct is selected from the group consisting of SEQ ID NOs: 88, 90-96, 98-100.

TABLE 6
Fusion Protein Constructs and Control Constructs

| Construct | SEQ ID | Construct | | |
|---|---|---|---|---|
| No | NO: | Name | Length | Sequence |
| 1 | 88 | JB1-SynBP1 | 110 | MGKDYYQTLGLARGASDEEIKRAYRRQALRYHPDK |
| | | | | NKEPGAEEKFKEIAEAYDVLSDPRKREIFDRYGEE |
| | | | | GLKGSDIGGGGSGGGGSGGGGSGGGGSAAAKDGIV |
| | | | | NGVKA |
| 2 | 89 | J(P330)- | 110 | MGKDYYQTLGLARGASDEEIKRAYRRQALRYHQDK |
| | | SynBP1 | | NKEPGAEEKFKEIAEAYDVLSDPRKREIFDRYGEE |
| | | | | GLKGSDIGGGGSGGGGSGGGGSGGGGSAAAKDGIV |
| | | | | NGVKA |
| 3 | 90 | JB1- | 120 | MGKDYYQTLGLARGASDEEIKRAYRRQALRYHPDK |
| | | 2XSynBP1 | | NKEPGAEEKFKEIAEAYDVLSDPRKREIFDRYGEE |
| | | | | GLKGSDIGGGGSGGGGSGGGGSGGGGSAAAKDGIV |
| | | | | NGVKAKDGLVNGVKA |
| 4 | 91 | SynBP1-JB1- | 123 | MKDGIVNGVKAEFMGKDYYQTLGLARGASDEEIKR |
| | | SynBP1 | | AYRRQALRYHPDKNKEPGAEEKFKEIAEAYDVLSD |
| | | | | PRKREIFDRYGEEGLKGSDIGGGGSGGGGSGGGGS |
| | | | | GGGGSAAAKDGLVNGVKA |
| 5 | 92 | SynBP2-JB1 | 90 | MWRQTRKDEFMGKDYYQTLGLARGASDEEIKRAYR |
| | | | | RQALRYHPDKNKEPGAEEKFKEIAEAYDVLSDPRK |
| | | | | REIFDRYGEEGLKGSDIAAA |
| 6 | 93 | JB6-SynBP1 | 111 | MVDYYEVLGVQRHASPEDLKKAYRKLALKWHPDKN |
| | | | | PENKEEAERKFKQVAEAYEVLSDAKKRDLYDKYGK |
| | | | | EGLNGGDIGGGGSGGGGSGGGGSGGGGSAAAKDGL |
| | | | | VNGVKA |
| 7 | 94 | JB13-SynBP1 | 110 | MGQDYYSVLGLTRNSEDAQLKQAYRRLALKHHPLK |
| | | | | SNEPSSAELFRQLAEAYDVLSDPMKRGIYDKFGEE |
| | | | | GLKGGDLGGGGSGGGGSGGGGSGGGGSAAAKDGLV |
| | | | | NGVKA |
| 8 | 95 | JB1-NAC32 | 348 | MGKDYYQTLGLARGASDEELKRAYRRQALRYHPDK |
| | | | | NKEPGAEEKFKELAEAYDVLSDPRKRELFDRYGEE |
| | | | | GLKGSDIGGGGSGGGGSGGGGSGGGGSAAAQVQLQ |
| | | | | QSGPGLVKPSQTLSLTCAISGDSVSSNSAAWNWIR |
| | | | | QSPSRGLEWLGRTFYRSKWYNDYAASVKSRITIDP |
| | | | | DTSKNQFSLQLNSVTPEDTAVYYCTRQNLAGPFDS |
| | | | | WGQGTLVTVSSGILGSGGGGSGGGGSGGGGSEIVM |
| | | | | TQSPGTLSLSPGERATLSCRASQSVSSNYLAWYQQ |
| | | | | KAGQAPRLLISGASSRATGIPDRFSGSGSGTDFTL |
| | | | | TISRLEPEDFAVYYCQQYGSSTAFGPGTKVDIK |
| 9 | 96 | JB1- | 347 | MGKDYYQTLGLARGASDEEIKRAYRRQALRYHPDK |
| | | Syn_scFv | | NKEPGAEEKFKEIAEAYDVLSDPRKREIFDRYGEE |
| | | | | GLKGSDIGGGGSGGGGSGGGGSGGGGSAAALQSVL |
| | | | | TQPPSVSGAPGQRVAISCTGTSSDIGTGYDVNWYQ |
| | | | | HLPGTAPRLLIYGNTYRPSGVPDRFSASTGSKSGT |
| | | | | SASLVITDLQAEDEGDYYCQSFDNSLRGSVFGGGT |
| | | | | KVTVLGEGKSSGSGSESKASEVQLVOSGGGVVKPG |
| | | | | GSLRLSCEASGFILSDYYMTWIRQAPGKGLEWLAV |
| | | | | IDITSSYTNYADSVKGRFTISRDNAKNSVYLQMNS |
| | | | | LRAEDTAVYYCARLESGFFDYWGQGTLVTVSS |
| 10 | 97 | JB1-QBP1 | 111 | MGKDYYQTLGLARGASDEEIKRAYRRQALRYHPDK |
| | | | | NKEPGAEEKFKEIAEAYDVLSDPRKREIFDRYGEE |
| | | | | GLKGSDIGGGGSGGGGSGGGGSGGGGSAAASNWKW |
| | | | | WPGIFD |
| 11 | 98 | JB1GGGS_ | 90 | MGKDYYQTLGLARGASDEEIKRAYRRQALRYHPDK |
| | | SynBP1 | | NKEPGAEEKFKEIAEAYDVLSDPRKREIFDRYGEE |
| | | | | GLKGSGGGGSKDGIVNGVKA |
| 12 | 99 | JB1EAAAK- | 90 | MGKDYYQTLGLARGASDEEIKRAYRRQALRYHPDK |
| | | SynBP1 | | NKEPGAEEKFKEIAEAYDVLSDPRKREIFDRYGEE |
| | | | | GLKGSEAAAKKDGIVNGVKA |
| 13 | 100 | JB1SynBP1 | 85 | MGKDYYQTLGLARGASDEEIKRAYRRQALRYHPDK |
| | | | | NKEPGAEEKFKEIAEAYDVLSDPRKREIFDRYGEE |
| | | | | GLKGSKDGIVNGVKA |
| 14 | 101 | JB1GGGGS- | 91 | MGKDYYQTLGLARGASDEEIKRAYRRQALRYHPDK |
| | | QBP1 | | NKEPGAEEKFKEIAEAYDVLSDPRKREIFDRYGEE |
| | | | | GLKGSGGGGSSNWKWWPGIFD |

II. Nucleic Acids Encoding Fusion Protein Constructs

According to another aspect of the invention, provided are isolated nucleic acids comprising a polynucleotide sequence selected from (a) a polynucleotide encoding the fusion protein of any of the foregoing embodiments, or (b) the complement of the polynucleotide of (a). The present invention provides isolated nucleic acids encoding fusion proteins comprising the J domain and α-synuclein-binding domain, and sequences complementary to such nucleic acid molecules encoding the fusion proteins, including homologous variants thereof. In another aspect, the invention encompasses methods to produce nucleic acids encoding the fusion proteins disclosed herein, and sequences complementary to the nucleic acid molecules encoding fusion proteins, including homologous variants thereof. The nucleic acid according to this aspect of the invention can be a pre-messenger RNA (pre-mRNA), messenger RNA (mRNA), RNA, genomic DNA (gDNA), PCR amplified DNA, complementary DNA (cDNA), synthetic DNA, or recombinant DNA.

In yet another aspect, disclosed is a method of producing a fusion protein comprising (a) synthesizing and/or assembling nucleotides encoding the fusion protein, (b) incorporating the encoding gene into an expression vector appropriate for a host cell, (c) transforming the appropriate host cell with the expression vector, and (d) culturing the host cell under conditions causing or permitting the fusion protein to be expressed in the transformed host cell, thereby producing the biologically-active fusion protein, which is recovered as an isolated fusion protein by standard protein purification methods known in the art. Standard recombinant techniques in molecular biology is used to make the polynucleotides and expression vectors of the present invention.

In accordance with the invention, nucleic acid sequences that encode the fusion proteins disclosed herein (or its complement) are used to generate recombinant DNA molecules that direct the expression of the fusion proteins in appropriate host cells. Several cloning strategies are suitable for performing the present invention, many of which is used to generate a construct that comprises a gene coding for a fusion protein of the present invention, or its complement. In some embodiments, the cloning strategy is used to create a gene that encodes a fusion protein of the invention, or their complement.

In certain embodiments, a nucleic acid encoding one or more fusion proteins is an RNA molecule, and can be a pre-messenger RNA (pre-mRNA), messenger RNA (mRNA), RNA, genomic DNA (gDNA), PCR amplified DNA, complementary DNA (cDNA), synthetic DNA, or recombinant DNA. In various embodiments, the nucleic acid is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA. In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from Rnases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly (A) sequence of between about 100 and about 1000 bases, between about 200 and about 500 bases, or between about 300 and about 400 bases. In one embodiment, the mRNA comprises a poly (A) sequence of about 65 bases, about 100 bases, about 200 bases, about 300 bases, about 400 bases, about 500 bases, about 600 bases, about 700 bases, about 800 bases, about 900 bases, or about 1000 or more bases. Poly(A) sequences can be modified chemically or enzymatically to modulate mRNA functionality such as localization, stability or efficiency of translation. As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

In certain embodiments, the nucleic acid sequence comprises a nucleotide sequence encoding the gene of interest (e.g., the fusion proteins comprising a J domain and a polyglutamine binding domain) within a nucleic acid cassette. The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. The cassette can be removed and inserted into a plasmid or viral vector as a single unit.

Illustrative ubiquitous expression control sequences suitable for use in particular embodiments include, but are not limited to, a cytomegalovirus (CMV) immediate early promoter, a viral simian virus 40 (SV40) (e.g early or late), a Moloney murine leukemia virus (MoMLV) LTR promoter, a Rous sarcoma virus (RSV) LTR, a herpes simplex virus (HSV) (thymidine kinase) promoter, H5, P7.5, and PI I promoters from vaccinia virus, an elongation factor 1-alpha (EFIa) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (b-KIN), the human ROSA 26 locus Orions et al, Nature Biotechnology 25, 1477-1482 (2007)), a Ubiquitin C promoter (UBC), a phosphogly cerate kinase-1 (PGK) promoter, a cytomegalovirus enhancer/chicken β-actin (CAG) promoter (Okabe et al. (1997) FEBS let. 407: 313-9), a b-actin promoter and a myeloproliferative sarcoma virus enhancer, negative control region deleted, dI587rev primer binding site substituted (MND) U3 promoter (Haas et al., Journal of Virology. 2003; 77(17): 9439-9450). In one embodiment, at least one element may be used with the polynucleotides described herein to enhance the transgene target specificity and expression (See e.g., Powell et al. (2015) Discovery Medicine 19(102):49-57, the contents of which are herein incorporated by reference in its entirety) such as promoters. Promoters for which promote expression in most tissues include, but are not limited to, human elongation factor la-subunit (EFIa), immediate-early cytomegalovirus (CMV), chicken β-actin (CBA) and its derivative CAG, the β glucuronidase (GUSB), or ubiquitin C (UBC). Tissue-specific expression elements can be used to restrict expression to certain cell types such as, but not limited to, nervous system promoters which can be used to restrict expression to neurons, astrocytes, or oligodendrocytes. Non-limiting example of tissue-specific expression elements for neurons include neuron-specific enolase (NSE), platelet-derived growth factor (PDGF), platelet-derived growth factor B-chain (PDGF-β), the synapsin (Syn), the methyl-CpG binding protein 2 (MeCP2), CaMKII, mGluR2, NFL, NFH, ηβ2, PPE, Enk and EAAT2 promoters. A non-limiting example of a tissue-specific expression elements for astrocytes include the glial fibrillary acidic protein (GFAP) and EAAT2 promoters. A non-limiting example of a tissue-specific expression element for oligodendrocytes include the myelin basic protein (MBP) promoter. Yu et al. (2011) Molecular Pain, 7:63, incorporated by reference in its entirety) evaluated the expression of eGFP under the CAG, EFIa, PGK and UBC promoters in rat DRG cells and primary DRG cells using lentiviral vectors and found that UBC showed weaker expression than the other 3 promoters and there was only 10-12% glia expression seen for all promoters. Soderblom et al. (E. Neuro 2015, incorporated by reference in its entirety) the expression of eGFP in AAV8 with CMV and UBC promoters and AAV2 with the CMV promoter after injection in the motor cortex. Intranasal administration of a plasmid containing a UBC or EFIa promoter showed a sustained airway expression greater than the expression with the CMV promoter (See e.g., Gill et al, (2001) Gene Therapy, Vol. 8, 1539-1546; incorporated by reference in its entirety). Husain et al. (2009) Gene Therapy, incorporated by reference in its entirety) evaluated a HβH construct with a hGUSB promoter, a HSV-1LAT promoter and a NSE promoter and found that the HβH construct showed weaker expression than NSE in mice brain. Passini and Wolfe (J. Virol. 2001, 12382-12392, incorporated by reference in its entirety) evaluated the long term effects of the HβH vector following an intraventricular injection in neonatal mice and found that there was sustained expression for at least 1 year. Low expression in all brain regions was found by Xu et al. (2001) Gene Therapy, 8, 1323-1332; incorporated by reference in its entirety) when NF-L and NF-H promoters were used as compared to the CMV-lacZ, CMV-luc, EF, GFAP, hENK, nAChR, PPE, PPE+wpre, NSE (0.3 kb), NSE (1.8 kb) and NSE (1.8 kb+wpre). Xu et al. found that the promoter activity in descending order was NSE (1.8 kb), EF, NSE (0.3 kb), GFAP, CMV, hENK, PPE, NFL and NFH. NFL is a 650 nucleotide promoter and NFH is a 920 nucleotide promoter which are both absent in the liver but NFH is abundant in the sensory proprioceptive neurons, brain and spinal cord and NFH is present in the heart. Scn8a is a 470 nucleotide promoter which expresses throughout the DRG, spinal cord and brain with particularly high expression seen in the hippocampal neurons and cerebellar Purkinje cells, cortex, thalamus and hypothalamus (See e.g., Drews et al. 2007 and Raymond et al. 2004; incorporated by reference in its entirety).

III. Vectors Comprising Nucleic Acids Encoding Fusion Proteins

Also provided is a vector comprising nucleic acid according to the invention. Such a vector preferably comprises additional nucleic acid sequences such as elements necessary for transcription/translation of the nucleic acid sequence encoding a phosphatase (for example promoter and/or terminator sequences). Said vectors can also comprise nucleic acid sequences coding for selection markers (for example an antibiotic) to select or maintain host cells transformed with said vector. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to an affected cell (e.g. neuronal cells) In one embodiment, the vector is an in vitro synthesized or synthetically prepared mRNA encoding a fusion protein comprising a J domain and a α-synuclein-binding domain. Illustrative examples of non-viral vectors include, but are not limited to mRNA, plasmids (e.g., DNA plasmids or RNA plasmids), transposons, cosmids, and bacterial artificial chromosomes.

Illustrative examples of vectors include, but are not limited to, a plasmid, autonomously replicating sequences, and transposable elements, e.g., piggyBac, Sleeping Beauty, Mosl, Tcl/mariner, Tol2, mini-Tol2, Tc3, MuA, Himar I, Frog Prince, and derivatives thereof. Additional Illustrative examples of vectors include, without limitation, plasmids, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Illustrative examples of viruses useful as vectors include, without limitation, retrovirus (including lentivirus), adenovirus, adeno-associated virus, herpesvirus (e.g., herpes simplex vims), poxvirus, baculovirus, papillomavirus, and papovavirus (e.g., SV40). Illustrative examples of expression vectors include, but are not limited to, pClneo vectors (Promega) for expression in mammalian cells; pLenti4/V 5-DEST™, pLenti6/V 5-DEST™, and pLenti6.2/V 5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

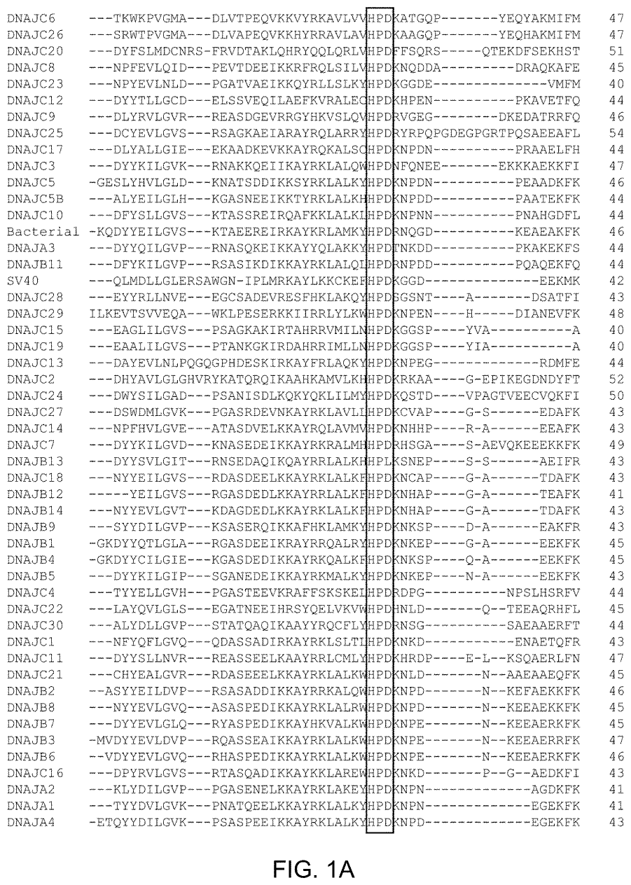

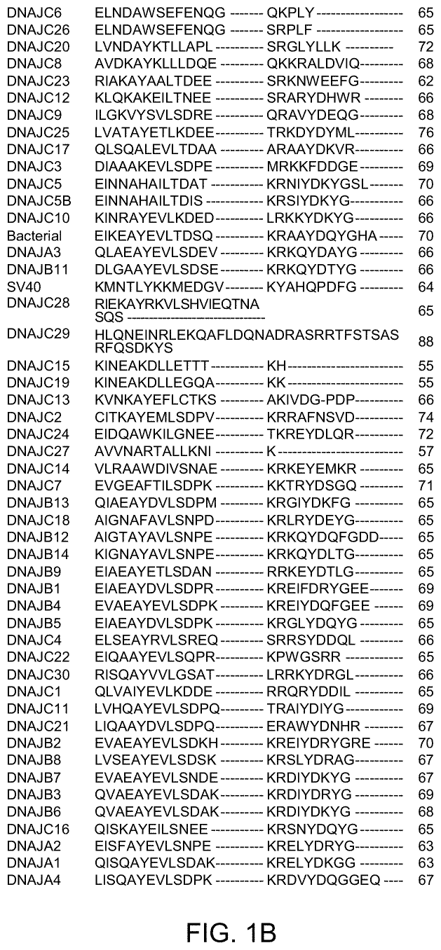

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Suitable recognition sites for the FLP recombinase include, but are not limited to: FRT (McLeod, et al., 1996), FI, F2, F3 (Schlake and Bode, 1994), FyFs (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme I Integrase, e.g., phi-c3I. The (pC3I SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by ϕ031 homodimers (Groth et al., 2000). The product sites, attL and attR, are effectively inert to further tpQA 1-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a fusion protein comprising a J domain and α-synuclein-binding domain. Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding fusion protein comprising a J domain and a polyglutamine-binding domain are introduced into a cell, e.g., a neuronal cell, by transducing the cell with a recombinant adeno-associated virus (rAAV), comprising the one or more polynucleotides. AAV is a small (^{˜}26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2 (described, for example, in U.S. Pat. No. 6,962,815B2, which is incorporated herein by reference in its entirety), AAV3, AAV4, AAV5 (described, for example, in U.S. Pat. No. 7,479,554B2, which is incorporated herein by reference in its entirety), AAV6, AAV7, AAV8 (described, for example, in U.S. Pat. No. 7,282,199B2, which is incorporated herein by reference in its entirety), AAV9 (described, for example, in U.S. Pat. No. 9,737,618B2, which is incorporated herein by reference in its entirety), AAV rh10 (described, for example, in U.S. Pat. No. 9,790,472B2, which is incorporated herein by reference in its entirety) or AAV 10. In one embodiment, the vector of the present invention is encapsulated into a capsid selected from the group consisting of AAV2, AAV5, AAV8, AAV9 and AAV rh10. In one embodiment, the vector is encapsulated in AAV2. In one embodiment, the vector is encapsulated in AAV5. In one embodiment, the vector is encapsulated in AAV8. In one embodiment, the vector is encapsulated in AAV9. In still one embodiment, the vector is encapsulated in AAV rh10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

IV. Delivery

In particular embodiments, one or more polynucleotides encoding a fusion protein comprising a J domain and α-synuclein-binding domain are introduced into a cell by non-viral or viral vectors. Illustrative methods of non-viral delivery of polynucleotides contemplated in particular embodiments include, but are not limited to: electroporation, sonoporation, lipofection, microinjection, biolistics, virosomes, liposomes, immunoliposomes, nanoparticles, poly cation or lipidnucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al., (2003) Gene Therapy. 10: 180-187; and Balazs et al., (20 W) Journal of Drug Delivery. 2011:1-12. Antibody-targeted, bacterially derived, non-living nanocell-based delivery is also contemplated in particular embodiments.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion), by intrathecal injection, intracerebroventricular injection or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding a fusion protein disclosed herein is administered directly to an organism for transduction of cells in vivo.

A viral vector, suitably packaged and formulated, can be delivered into the central nervous system (CNS) via intrathecal delivery. For example, adeno-associated viral vectors can be delivered using methods described in U.S. Ser. No. 15/771,481, which is incorporated herein by reference in its entirety.

Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

In various embodiments, one or more polynucleotides encoding a fusion protein disclosed herein are introduced into a cell, for example, a neuronal cell or neuronal stem cell, by transducing the cell with a retrovirus, e.g., lentivirus, comprising the one or more polynucleotides. As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus. As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex vims (HSV) (thymidine kinase) promoters. In certain embodiments, lentiviral vectors are produced according to known methods. See e.g., Kutner et al., BMC Biotechnol. 2009; 9:10. Doi: 10.1186/1472-6750-9-10; Kutner et al., Nat. Protoc. 2009; 4(4):495-505. Doi: 10.1038/nprot.2009.22.

According to certain specific embodiments contemplated herein, most or all of the viral vector backbone sequences are derived from a lentivirus, e.g., HIV-I. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a fusion protein disclosed herein are introduced into a target cell by transducing the cell with an adenovirus comprising the one or more polynucleotides. Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad Ela, E1 b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7: 1083-9 (1998)).

In various embodiments, one or more polynucleotides encoding a fusion protein of the invention are introduced into the target cell of a subject by transducing the cell with a herpes simplex virus, e.g., HSV-I, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which is incorporated by reference herein in its entirety.

V. Cells Expressing the Fusion Protein

In yet another aspect, the invention provides for cells expressing the fusion proteins described herein. Cells can be transfected with a vector encoding the fusion protein as described herein above. In one embodiment, the cell is a prokaryotic cell. In another embodiment, the cell is a eukaryotic cell. In still another embodiment, the cell is a mammalian cell. In a particular embodiment, the cell is a human cell. In another embodiment, the cell is a human cell that is derived from a patient that suffers from, or is at risk of suffering from, a α-synuclein-mediated disorder including, but not limited to, ALS, FTD and Alzheimer's Disease. The cell can be a neuronal cell or a muscle cell.

Cells expressing the fusion protein can be useful in producing the fusion protein. In this embodiment, the cells are transfected with a vector overexpressing the fusion protein. The fusion protein may optionally contain an epitope, for example, a human Fc domain or a FLAG epitope, as described herein above, that would facilitate the purification (using a Protein A- or anti-FLAG antibody column, respectively). The epitope may be connected to the rest of the fusion protein via a linker or a protease substrate sequence such that, during or after purification, the epitope can be removed from the fusion protein.

Cells expressing the fusion protein can also be useful in a therapeutic context. In one embodiment, cells are collected from a patient in need of therapy (e.g., a patient who suffers from or is at risk of suffering from a α-synuclein-mediated disorder). In one embodiment, the cells are neuronal cells. Collected cells are then transfected with a vector expressing the fusion protein. The transfected cells can then be processed to enrich or select for transfected cells. The transfected cells can also be treated to differentiate into a different type of cell, for example, a neuronal cell. After processing, the transfected cells can be administered to the patient. In one embodiment, the cells are administered by directed injection into the central nervous system by intrathecal injection, intracranial injection or intracerebroventricular injection.

In an alternative embodiment, cells expressing a secreted form of the fusion protein can be used. For example, fusion protein constructs can be designed having a signal sequence on the N-terminal end. Representative signal sequences are shown below in Table 7.

TABLE 7
Representative Signal Sequences

| SEQ ID NO: | Length | SEQUENCE |
|---|---|---|
| 102 | 17 | MGVKVLFALICIAVAEA |
| 103 | 19 | MAPVQLLGLLVLFLPAMRC |
| 104 | 19 | MAVLGLLFCLVTFPSCVLS |

Therefore, in one embodiment, the fusion protein comprises a signal sequence and a fusion protein, wherein the signal sequence is selected from the group consisting of SEQ ID NOs: 102-104, and the fusion protein is selected from the group consisting of SEQ ID NOs: 88, 90-96, 98-100. In another embodiment, the fusion protein comprises the signal sequence of SEQ ID NO: 102, and the fusion protein selected from the group consisting of SEQ ID NOs: 88, 90-96, 98-100. In another embodiment, the fusion protein comprises the signal sequence of SEQ ID NO: 103, and the fusion protein selected from the group consisting of SEQ ID NOs: 88, 90-96, 98-100. In another embodiment, the fusion protein comprises the signal sequence of SEQ ID NO: 104, and the fusion protein selected from the group consisting of SEQ ID NOs: 88, 90-96, 98-100. Cells expressing the fusion protein constructs with the signal sequence can be administered to a subject, for example a human subject (e.g., a patient having or at risk of suffering from a α-synuclein disorder). The fusion protein is secreted from the cells, which help reduce α-synuclein protein aggregation and/or associated cytotoxicity.

As described herein above, in certain embodiments, the fusion protein can further comprise a cell-penetrating peptide. A cell expressing a fusion protein comprising a signal sequence and a cell-penetrating peptide would be capable of secreting the fusion protein, devoid of the signal sequence. The secreted fusion protein, also comprising the cell-penetrating peptide, would then be capable of entering nearby cells, and have the potential to reduce aggregation and/or cytotoxicity mediated by α-synuclein proteins in those cells.

VI. Methods of Use

In another aspect, the invention provides a method for achieving a beneficial effect in disorders and/or in a α-synuclein disorder, disorder or condition mediated by α-synuclein aggregation. The α-synuclein disorder is selected from the group consisting of amyotrophic lateral sclerosis (ALS), frontotemporal dementia (FTD), Parkinsonism, Huntington's disease, Alzheimer's disease, hippocampal sclerosis, and dementia with Lewy's bodies.

In some embodiments, the invention provides methods for treating a subject, such as a human, with a α-synuclein disease, disorder or condition comprising the step of administering to the subject a therapeutically- or prophylactically-effective amount of a fusion protein, a nucleic acid encoding such fusion protein, or a viral vector encoding such fusion protein described herein, wherein said administration results in the improvement of one or more biochemical or physiological parameters or clinical endpoints associated with the α-synuclein disease, disorder or condition.

In other embodiments, the invention provides for a method of reducing aggregation of α-synuclein in a cell. The cell can be a cultured cell or an isolated cell. The cell can also be from a subject, for example, a human subject. In one embodiment, the cell is in the central nervous system of the human subject. In another embodiment, the human subject is suffering from, or is at risk of suffering from a α-synuclein disorder disease, including, but not limited to, amyotrophic lateral sclerosis (ALS), frontotemporal dementia (FTD) and Alzheimer's Disease. In one particular embodiment, the α-synuclein disorder is amyotrophic lateral sclerosis.

Aggregation of α-synuclein proteins can be detected in a number of ways. In one example, aggregated α-synuclein proteins can be detected in ELISA with antibody specifically recognizing α-synuclein conformation. Reduction of aggregation of α-synuclein proteins can also be detected directly in the cell, for example, using immunofluorescence microscopy with labeled reagents detecting the α-synuclein protein (see, for example, Ding et al., (2015) Oncotarget, 6: 24178-24191; Chou et al., (2015) Hum. Mol. Genet. 24:5154-5173, and Example 1). In certain embodiments, a greater reduction of α-synuclein polypeptide levels when compared with controls indicates a higher potency.

Therefore, in one embodiment, the method comprises contacting the cell with an amount of the fusion protein or a nucleic acid, vector, or viral particle encoding the fusion protein effective to reduce aggregation of α-synuclein proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

As shown below in Example 1, expression of fusion proteins comprising a J domain and a α-synuclein-binding domain has been found to reduce the overall level of α-synuclein-containing reporter constructs. As such, in another embodiment, the method comprises contacting the cell with an amount of the fusion protein, a cell expressing the fusion protein, a nucleic acid, vector, or viral particle encoding the fusion protein effective to reduce the level of α-synuclein proteins by at least 10%, for example, at least 15%, at least 20%, at least 30%, at least 40%, at least 50%, at least 60%, at least 70%, at least 75%, at least 80%, at least 90%, at least 95%, at least 98%, at least 99%, when compared with an untreated or control cell.

VII. Pharmaceutical Compositions

The compositions contemplated herein may comprise one or more fusion protein comprising a J domain and α-synuclein-binding domain, polynucleotides encoding such fusion proteins, vectors comprising same, genetically modified cells, etc., as contemplated herein. Compositions include, but are not limited to pharmaceutical compositions. A “pharmaceutical composition” refers to a composition formulated in pharmaceutically acceptable or physiologically acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein “pharmaceutically acceptable carrier”, “diluent” or “excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

VIII. Dosages

The dosage of the compositions (e.g., a composition including a fusion protein construct, nucleic acid or gene therapy viral particle) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. The compositions described herein can be administered initially in a suitable dosage that can be adjusted as required, depending on the clinical response. In some aspects, the dosage of a composition is a prophylactically or a therapeutically effective amount.

IX. Kits

Kits including (a) a pharmaceutical composition including a fusion protein construct, nucleic acid encoding such fusion protein, or viral particle encompassing such nucleic acid that reduces aggregation of α-synuclein proteins in a cell or subject described herein, and (b) a package insert with instructions to perform any of the methods described herein are contemplated. In some aspects, the kit includes (a) a pharmaceutical composition including a composition described herein that reduces the aggregation of α-synuclein proteins in a cell or subject described herein, (b) an additional therapeutic agent, and (c) a package insert with instructions to perform any of the methods described herein.

### EXAMPLES

To test whether J domains can be specifically engineered to facilitate the proper folding of aggregated proteins, we designed and tested a number of fusion protein constructs designed to target α-synuclein proteins.

### Example 1: Fusion Protein Design

A. Methods

### General Techniques and Materials

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al., eds., 1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory, 1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4th edition, John Wiley & Sons, Somerset, N J, 2000, the contents of which are incorporated in their entirety herein by reference. HEK-293 cells (human embryonic kidney cells) were purchased from the American Type Culture Collection (Manassas, Va.). Anti-FLAG antibody was purchased from Thermo Fisher Scientific. Rabbit anti-GFP antibody was purchased from GenScripts (Piscataway, N.J.). For ease of purification and characterization, some of the fusion protein constructs used in this Example 1 contain, in addition to the sequences provided in SEQ ID NOs: 88-101, the FLAG epitope of SEQ ID NO:78 at either the C-terminus or N-terminus of the protein, in addition to a short linker sequence.

### Expression and Detection of Proteins in HEK293 Cells

Expression vector plasmids encoding various protein constructs were transfected into HEK293 cells with Lipofectamine 3000 transfection reagent (Thermo Fisher Scientific). Cell lysates were analyzed for expressed proteins using immunoblot assays. Samples of culture media were centrifuged to remove debris prior to analysis. Cells were lysed in a lysis buffer (10 mM Tris-HCl, pH 8.0, 150 mM NaCl, 10 mM EDTA, 2% SDS) containing 2 mM PMSF and protease cocktail (Complete Protease Inhibitor Cocktail; Sigma). After brief sonication, the samples were analyzed for expressed proteins using immunoblot assays. For immunoblot analysis, samples were boiled in an SDS-sample buffer and run on polyacrylamide electrophoresis. Thereafter, the separated protein bands were transferred to a PVDF membrane.

Expressed proteins were detected using a chemiluminescent signal. Briefly, blots were reacted with a primary antibody capable of binding the particular epitope (e.g., anti-α-synuclein antibody). After rinsing away the unreacted primary antibody, a secondary, enzyme-linked antibody (e.g., HRP-linked anti-IgG antibody) was allowed to react with the primary antibody molecules bound to the blots. Following rinsing, a chemiluminescent reagent was added, and the resultant chemiluminescent signals in the blots were captured on X-ray film.

### BFA/MG132 Incubation

Expression vector plasmids encoding various protein constructs were transfected into HEK293 cells with Lipofectamine 3000 transfection reagent (Thermo Fisher Scientific). One day after transfection, culture medium was replaced with fresh medium containing bafilomycin al (BFA) or MG132, and cells were incubated for additional 48 hours. Cells were lysed in lysis buffer (20 mM Tris, pH 7.4, 150 mM NaCl, 1 mM EDTA, 0.1% NP-40) supplemented with protease inhibitor cocktail (Complete Protease Inhibitor Cocktail; Sigma) and 2 mM PMSF. After brief sonication, cell lysates were analyzed in ELISA or immunoblotting assay.

### ELISA (Enzyme-Linked Immunosorbent Assay)

The aggregation status of expressed α-synuclein was monitored with α-synuclein aggregate-specific antibody Syn-O4 (BioLegend). Syn-O4 is a conformation-specific monoclonal antibody that recognizes specifically α-synuclein aggregates but not the soluble, monomeric form of the protein⁵⁸. Microtiter plate wells were coated with Syn-O4 overnight at 4° C., and after coating, the wells were washed three times with PBS, blocked for one hour with PBS containing 1% BSA, and washed again with PBS.

After the expression, cultured cells were lysed in lysis buffer (20 mM Tris, —HCl, pH7.4, 150 mM NaCl, 1 mM EDTA, 0.1% NP-40) containing 2 mM PMSF and protease cocktail, followed by brief sonication. After removal of debris by centrifuge, the cell lysate was added to the well and incubated for overnight at 4 C. The wells were then washed three times with PBS, incubated with HRP-linked anti-alpha-synuclein antibody in PBS containing 1% BSA for one hour, and washed again three times with PBS. Finally, peroxidase activity was assayed with 0.01 mg/ml 3′,3′,5′,5′-tetramethylbenzidine (TMB) and 0.01% H₂O₂ in a buffer of 0.1 M sodium acetate (pH 6.0). After an equal volume of 1 M HCl was added, the optical density was determined at 450 nm. Reporter Constructs

We first investigated whether the fusion proteins targeting α-synuclein ameliorates its aggregation in cultured cells. To this end, we generated two constructs expressing the wildtype α-synuclein, as well as a mutant form (containing an A53T substitution) known to be associated with familial Parkinson's disease (See Table 8 below). HEK293 cells were cultured and transfected with the plasmids encoding the wildtype (SEQ ID NO: 105) or mutant (SEQ ID NO: 106) α-synuclein.

TABLE 8
Synuclein Constructs

| Construct | | | |
|---|---|---|---|
| Name | SEQ ID NO: | Length | Sequence |
| alpha- | 105 | 140 | MDVFMKGLSKAKEGVVAAAEKTKQGVAEAAGKTKEGVL |
| synuclein | | | YVGSKTKEGVVHGVATVAEKTKEQVTNVGGAVVTGVTAV |
| | | | AQKTVEGAGSIAAATGFVKKDQLGKNEEGAPQEGILEDM |
| | | | PVDPDNEAYEMPSEEGYQDYEPEA |
| alpha- | 106 | 140 | MDVFMKGLSKAKEGVVAAAEKTKQGVAEAAGKTKEGVL |
| synuclein | | | YVGSKTKEGVVHGVTTVAEKTKEQVTNVGGAVVTGVTAV |
| (A53T) | | | AQKTVEGAGSIAAATGFVKKDQLGKNEEGAPQEGILEDM |
| | | | PVDPDNEAYEMPSEEGYQDYEPEA |

### Fusion Protein Constructs

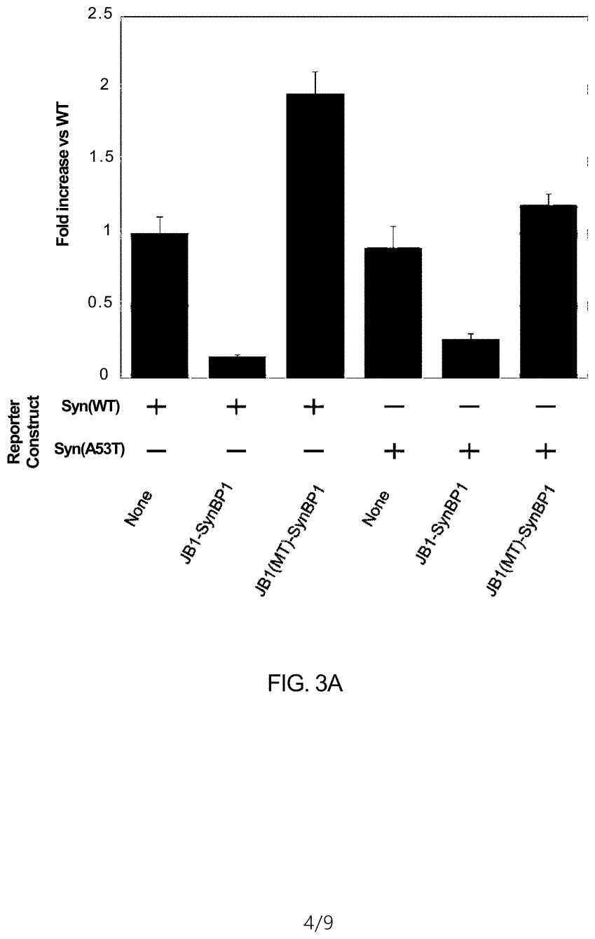

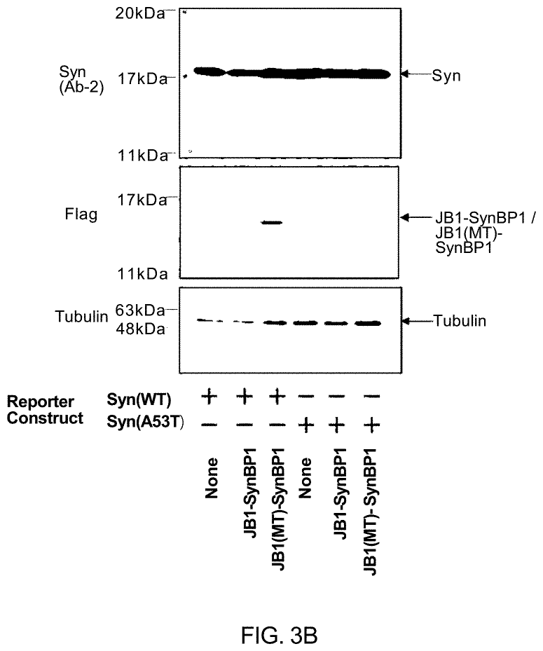

To determine whether J domains could be used to reduce α-synuclein aggregation, an initial experiment was first conducted by co-expression wildtype or mutant synuclein with a fusion protein comprising a J-domain sequence derived from a Hsp40 J-domain protein (from human DnaJB1), conjugated to the synuclein-binding peptide SynBP1 (see Construct 1 in Table 9 below). As shown in FIG. 3A, expression of either wildtype or mutant (A53T) α-synuclein in HEK293 cells results in the appearance of aggregated α-synuclein as detectable by ELISA on cell lysates (see bars 1 and 4). Co-expression of Construct 1 results in a dramatic reduction in the amount of detectable aggregated α-synuclein (bars 2 and 5). However, co-expression of a mutant variant of Construct 1, containing a P33Q mutation within the highly conserved HPD motif within the J domain, results in higher overall aggregation (see bars 3 and 6), suggesting that the mechanism of reduced aggregation is through the HSP70 system. Western blot analysis of cell lysates was performed using an anti-α-synuclein antibody to determine whether the fusion protein affected the overall level of α-synuclein. As show in FIG. 3B, all cells had relatively similar levels of α-synuclein, suggesting that the reduction in aggregated α-synuclein is not due solely to degradation of α-synuclein.

TABLE 9
Fusion Protein Constructs and Controls

| Construct | SEQ ID | | |
|---|---|---|---|
| No | NO: | Construct Name | Notes |
| 1 | 88 | JB1-SynBP1 | J domain from human DnaJB1 fused to 4XGGGGS |
| | | | linker, in turn, fused on its C-terminus to SynBP1 |
| 2 | 89 | J(P33Q)-SynBP1 | Same as construct 1, but containing a P33Q |
| | | | mutation within the J domain |
| 3 | 90 | JB1-2XSynBP1 | J domain from human DnaJB1 fused to 4XGGGGS |
| | | | linker, in turn, fused on its C-terminus to two |
| | | | tandem copies of SynBP1 |
| 4 | 91 | SynBP1-JB1-SynBP1 | Construct with two copies of SynBP1, arranged on |
| | | | either side of the J domain from human DnaJB1 |
| 5 | 92 | SynBP2-JB1 | J domain from human DnaJB1 fused on its N- |
| | | | terminus to SynBP2 |
| 6 | 93 | JB6-SynBP1 | J domain from human DnaJB6 fused to 4XGGGGS |
| | | | linker, in turn, fused on its C-terminus to SynBP1 |
| 7 | 94 | JB13-SynBP1 | J domain from human DnaJB13 fused to 4XGGGGS |
| | | | linker, in turn, fused on its C-terminus to SynBP1 |
| 8 | 95 | JB1-NAC32 | J domain from human DnaJB1 fused to 4XGGGGS |
| | | | linker, in turn, fused on its C-terminus to the scFv |
| | | | NAC32 |
| 9 | 96 | JB1-Syn_scFv | J domain from human DnaJB1 fused to 4XGGGGS |
| | | | linker, in turn, fused on its C-terminus to the |
| | | | Syn_scFv |
| 10 | 97 | JB1-QBP1 | J domain from human DnaJB1 fused to 4XGGGGS |
| | | | linker, in turn, fused on its C-terminus to QBP1 |
| 11 | 98 | JB1GGGS-SynBP1 | J domain from human DnaJB1 fused on its C- |
| | | | terminus to SynBP1 (with flexible GGGGS linker) |
| 12 | 99 | JB1EAAAK-SynBP1 | J domain from human DnaJB1 fused on its C- |
| | | | terminus to SynBPI (with rigid EAAAK linker) |
| 13 | 100 | JB1SynBP1 | J domain from human DnaJB1 fused on its C- |
| | | | terminus to SynBPI (no linker) |
| 14 | 101 | JB1GGGGS-QBP1 | J domain from human DnaJB1 fused on its C- |
| | | | terminus to QBP1 (with flexible GGGGS linker) |

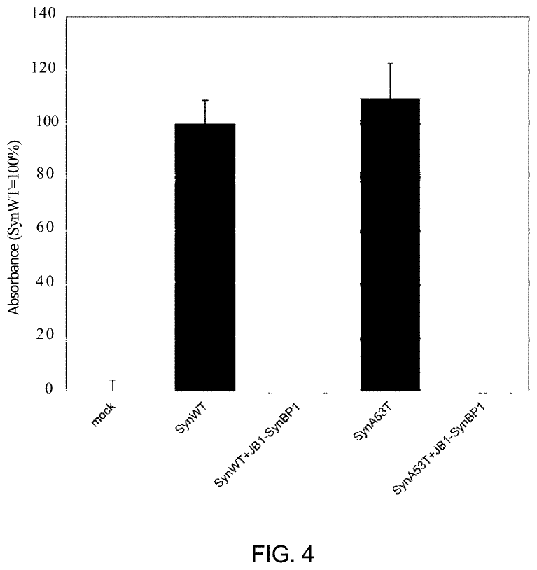

α-synuclein is known to be secreted. We therefore investigated whether the fusion protein constructs were effective in reducing the amount of secreted α-synuclein. Culture media were collected from cells expressing wildtype (bars 2 and 3) or mutant (bars 4 and 5) α-synuclein, alone or also expressing Construct 1 (JB1-SynBP1; bars 3 and 5) and tested for the level of secreted α-synuclein by ELISA analysis. As shown in FIG. 4, when expressed without the fusion protein construct, both wildtype and mutant (A53T) α-synuclein forms are secreted (bars 2 and 4, respectively). However, cells co-expressing Construct 1 have no detectable α-synuclein secreted into the culture media, as measured by ELISA (bars 3 and 5 for wildtype and mutant α-synuclein, respectively).

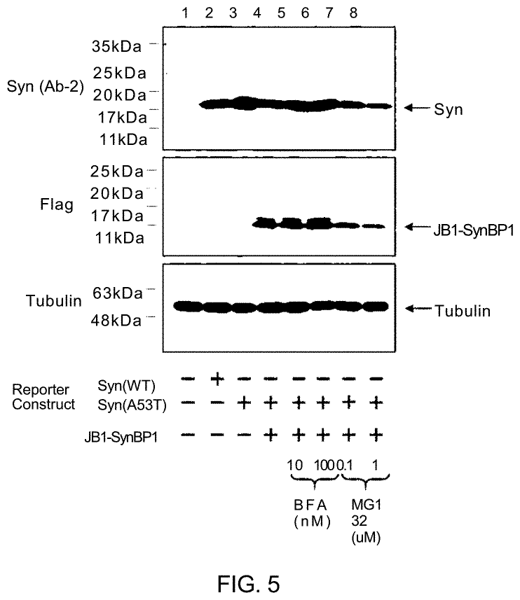

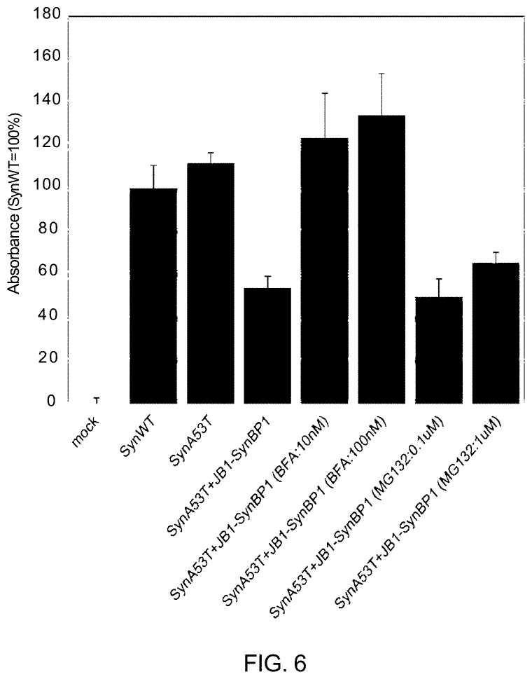

We next investigated the mechanism of the fusion protein in reducing aggregation. HEK293 cells were transfected with wildtype or mutant (A53T) α-synuclein, either alone or with Construct 1 (JB1-SynBP1), as well as with BFA (Bafilomycin A1, an inhibitor of the late phase of autophagy) or MG132 (proteasome inhibitor). FIGS. 5 and 6 show the results, which is also summarized below in Table 10 (values are normalized to the wildtype alone control). As can be seen, expression of construct 1 in cells also expressing the mutant (A53T) α-synuclein results in a moderate reduction in total α-synuclein (from 134.9% to 99%), but a more pronounced reduction in aggregated α-synuclein (from 111.5% to 53.9%). Treatment of these cells with BFA at either 10 nM or 100 nM resulted in restoration of total and aggregated α-synuclein to levels matching cells expressing mutant α-synuclein alone (i.e., not also transfected with the fusion protein construct 1). Bafilomycin A1 is a potent inhibitor of late phase autophagy, suggesting that the fusion protein constructs exert their effects via chaperon-mediated autophagy.

In contrast, treatment of cells with MG132, a proteasome inhibitor, had little effect on the total or aggregated levels of α-synuclein.

TABLE 10
Effect of inhibitors in fusion protein-mediation reduction of α-synuclein
aggregation

| | Fusion | | | |
|---|---|---|---|---|
| | Protein | | Total α- | Aggregated |
| α-synuclein | Construct | Inhibitor | synuclein | α-synuclein |
| None | None | None | 0% | 0% |
| Wildtype | None | None | 100.0% | 100% |
| Mutant (A53T) | None | None | 134.9% | 111.5% |
| Mutant (A53T) | JB1-SynBP1 | None | 99.0% | 53.9% |
| Mutant (A53T) | JB1-SynBP1 | BFA 10 nM | 104.1% | 123.4% |
| Mutant (A53T) | JB1-SynBP1 | BFA 100 nM | 123.9% | 134.0% |
| Mutant (A53T) | JB1-SynBP1 | MG132 | 86.9% | 49.7% |
| | | 0.1 μM | | |
| Mutant (A53T) | JB1-SynBP1 | MG132 1 μM | 60.0% | 65.4% |

Additional constructs were tested for the ability to reduce mutant (A53T) synuclein aggregation. Table 11 below shows that a number of other constructs comprising different synuclein-binding domains were capable of reducing α-synuclein aggregation. Interestingly, Construct 10, a fusion protein construct comprising QBP1, which was previously shown to bind to polyglutamine repeats, also showed the ability to potently reduce α-synuclein aggregation.

TABLE 11
Effect of fusion protein constructs containing various α-synuclein-
binding domains in reducing aggregation

| | Fusion Protein | | Aggregated α- |
|---|---|---|---|
| α-synuclein | Construct | Construct | synuclein |
| None | None | None | 0% |
| Wildtype | None | None | 100% |
| Mutant (A53T) | None | None | 78.9% |
| Mutant (A53T) | JB1-NAC32 | 8 | 57.3% |
| Mutant (A53T) | JBl-Syn_scFv | 9 | 46.3% |
| Mutant (A53T) | JB1-QBP1 | 10 | 28.5% |

To further optimize the arrangement of J domains and α-synuclein-binding domains, additional constructs were tested. Table 12 below shows the ability of constructs either employing an alternate J domain (Construct 6, using the J domain derived from DnaJB6; Construct 7, using the J domain from DnaJB13), or different linkers between the J domain and α-synuclein-binding domains.

TABLE 12
Effect of fusion protein constructs containing various α-synuclein-binding
domains in reducing aggregation

| | Fusion Protein | | | Aggregated |
|---|---|---|---|---|
| α-synuclein | Construct | Construct | Linker | α-synuclein |
| None | None | None | None | 0% |
| Wildtype | None | None | None | 100% |
| Mutant (A53T) | None | None | None | 113.2% |
| Mutant (A53T) | JB1-SynBP1 | 1 | (GGGGS)₄ | 82.0% |
| Mutant (A53T) | JB6-SynBP1 | 6 | (GGGGS)₄ | 65.2% |
| Mutant (A53T) | JB13-SynBP1 | 7 | (GGGGS)₄ | 116.1% |
| Mutant (A53T) | JB1SynBP1 | 13 | None | 18.0% |
| Mutant (A53T) | JB1GGGGSSynBP1 | 11 | GGGGS | 28.6% |
| Mutant (A53T) | JB1EAAAKSynBP1 | 12 | EAAAK | 46.2% |

Next, experiments were conducted to determine whether expression of various fusion protein constructs had an effect on the level of phosphorylated α-synuclein. Table 13 below shows that expression of various fusion protein constructs had profound effects on the level of the mutant (A53T) α-synuclein phosphorylated at Ser129.

TABLE 13
Effect of co-expressing various fusion protein constructs the level of
phosphorylated α-synuclein (Ser129).

| | Fusion Protein | | Phosphorylated |
|---|---|---|---|
| α-synuclein | Construct | Construct | α-synuclein |
| None | None | None | 0% |
| Wildtype | None | None | 100% |
| Mutant (A53T) | None | None | 92.1% |
| Mutant (A53T) | JB1-SynBP1 | 1 | 51.3% |
| Mutant (A53T) | JB1SynBP1 | 13 | 4.5% |
| Mutant (A53T) | JB1GGGGSSynBP1 | 11 | 19.1% |
| Mutant (A53T) | JB1EAAAKSynBP1 | 12 | 23.1% |

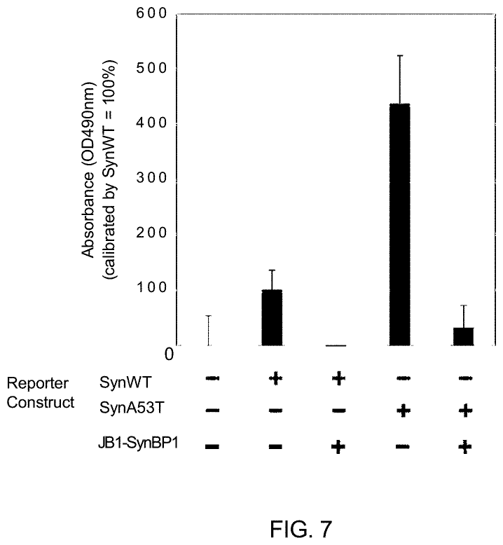

Finally, experiments were conducted to determine whether expression of various fusion protein constructs are able to ameliorate cytotoxicity by expression of mutant (A53T) α-synuclein. Although expression of either wild-type (WT) or mutant (A53T) α-synuclein did not induce significant cytotoxicity in HEK293 cells (data not shown), cytotoxic effect caused by α-synuclein was observed in U-87 MG cells, as evidenced by increased LDH activity in culture medium collected from U87-MG cells at 7-day after infection as measured by the LDH-Cytotox™ assay kit (BioLegend, San Diego, Calif., USA) (FIG. 7). Co-expression with JB1-SynBP1 (Construct 1) strongly suppressed cytotoxic effect of α-synuclein. We interpret these results to mean that the fusion protein construct is capable of reducing α-synuclein-mediated cytotoxicity.

### Example 2: AAV Vectors Encoding Fusion Protein Constructs

An exemplary gene therapy vector is constructed by an AAV9 vector bearing a codon-optimized cDNA encoding the fusion protein constructs of Table 6, specifically constructs 1, 3, 4, and 5, under the control of a CAG promoter, containing the cytomegalovirus (CMV) early enhancer element and the chicken beta-actin promoter. The cDNA encoding JB1-SynBP1 is located downstream of the Kozak sequence and is polyadenylated by the bovine growth hormone polyadenylation (BGHpA) signal. The entire cassette is flanked by two non-coding terminal inverted sequences of AAV-9.

Recombinant AAV vector is prepared using a baculovirus expression system similar to that described above (Urabe et al., 2002, Unzu et al., 2011 (reviewed in Kotin, 2011)). Briefly, three recombinant baculoviruses, one encoding REP for replication and packaging, one encoding CAP-5 for the capsid of AAV9, and one having an expression cassette is used to infect SF9 insect cells. Purification is performed using AVB Sepharose high speed affinity media (GE Healthcare Life Sciences, Piscataway, N.J.). Vectors are titrated using QPCR with the primer-probe combination for the transgene and titers are expressed as genomic copies per ml (GC/ml). The titer of the vector is approximately between 8×10¹³ to 2×10¹⁴ GC/ml.

### Example 3: Testing of Efficacy in a Mouse Model of PD

Vectors prepared above are tested in a mouse model of PD. A useful model includes the mouse model described in Fares et al., (2006) Proc. Natl. Acad. Sci. USA, 113:E912-E921, which was generated by expression of human α-synuclein in a SNCA⁻/⁻ background, which results in hyperphosphorylated (at S129) and ubiquitin-positive LB-like inclusions in primary neurons, which grow in size and incorporate soluble proteins.

To test the effect of the test compound or known compound described in the application in an animal model, the different AAV viral particles containing vectors encoding the fusion proteins and corresponding controls are administered to the transgenic animal. In one embodiment, the viral particles are administered by tail vein injection. In another embodiment, the viral particles are administered by intramuscular injection. In still another embodiment, the particles are administered by intracranial injection, for example as described in Stanek et al., (2014) Hum. Gene. Ther. 25:461-474.

After administration, disease progression is monitored and compared with control injected mice. In one embodiment, development of PD-like inclusion bodies is evaluated in the animal. Several other animal models that replicate PD-like pathology can also be used.

### Other Aspects

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An isolated fusion protein comprising a J domain of a J protein and a α-synuclein-binding domain.

2. The fusion protein of claim 1, wherein the J domain of a J protein is of eukaryotic origin.

3. The fusion protein of claim 1, wherein the J domain of a J protein is of human origin.

4. The fusion protein of claim 1, wherein the J domain of a J protein is cytosolically localized.

5. The fusion protein of claim 1, wherein the J domain of a J protein is selected from the group consisting of SEQ ID Nos: 1-50.

6. The fusion protein of claim 1, wherein the J domain comprises the sequence selected from the group consisting of SEQ ID NOs: 1, 5, 6, 10, 16, 24, 25, 31, and 49.

7. The fusion protein of claim 1, wherein the J domain comprises the sequence of SEQ ID NO: 5.

8. The fusion protein of claim 1, wherein the J domain comprises the sequence of SEQ ID NO: 10.

9. The fusion protein of claim 1, wherein the J domain comprises the sequence of SEQ ID NO: 16.

10. The fusion protein of claim 1, wherein the J domain comprises the sequence of SEQ ID NO: 25.

11. The fusion protein of claim 1, wherein the J domain comprises the sequence of SEQ ID NO: 31.

12. The fusion protein of claim 1, wherein the α-synuclein-binding domain has a K_{D} for α-synuclein of 1 μM or less, for example, 300 nM or less, 100 nM or less, 30 nM or less, 10 nM or less when measured using an ELISA assay.

13. The fusion protein of claim 12, wherein the α-synuclein-binding domain comprises the sequence selected from the group consisting of SEQ ID NOs: 51-64.

14. The fusion protein of claim 12, wherein the α-synuclein-binding domain comprises the sequence of SEQ ID NO: 51.

15. The fusion protein of claim 12, wherein the α-synuclein-binding domain comprises the sequence of SEQ ID NO: 52.

16. The fusion protein of claim 12, wherein the α-synuclein-binding domain comprises the sequence of SEQ ID NO: 63.

17. The fusion protein of claim 12, wherein the α-synuclein-binding domain comprises the sequence of SEQ ID NO: 64.

18. The fusion protein of claim 1, comprising a plurality of α-synuclein-binding domains.

19. The fusion protein of claim 18, consisting of two α-synuclein-binding domains.

20. The fusion protein of claim 18, consisting of three α-synuclein-binding domains.

21. The fusion protein of claim 1, comprising one of the following constructs:

| | |
|---|---|
| i. | DNAJ-X-S, |
| ii. | DNAJ-X-S-X-S, |
| iii. | DNAJ-X-S-X-S-X-S, |
| iv. | S-X-DNAJ, |
| v. | S-X-S-X-DNAJ, |
| vi. | S-X-S-X-S-X-DNAJ, |
| vii. | S-X-DNAJ-X-S, |
| viii. | S-X-DNAJ-X-S-X-S, |
| ix. | S-X-S-X-DNAJ-X-S-X-S-X-S, |
| x. | S-X-S-X-S-X-DNAJ-X-S, |
| xi. | S-X-S-X-S-X-DNAJ-X-S-X-S, |
| xii. | S-X-S-X-S-X-DNAJ-X-S-X-S-X-S, |
| xiii. | DnaJ-X-DnaJ-X-S-X-S, |
| xiv. | S-X-DnaJ-X-DnaJ, |
| | and |
| xv. | S-X-S-X-DnaJ-X-DnaJ, |

wherein,
S is a α-synuclein-binding domain,
DNAJ is a J domain of a J protein, and
X is an optional linker.

22. The fusion protein of claim 21, wherein the fusion protein comprises the J domain sequence of SEQ ID NO: 5 and the α-synuclein-binding domain sequence of SEQ ID NO: 51.

23. The fusion protein of claim 21, wherein the fusion protein comprises the J domain sequence of SEQ ID NO: 5 and two copies of the α-synuclein-binding domain sequence of SEQ ID NO: 51.

24. The fusion protein of claim 1, wherein the fusion protein comprises the sequence selected from the group consisting of SEQ ID NOs: 88, 90-96, 98-100.

25. The fusion protein of claim 24, wherein the fusion protein comprises the sequence of SEQ ID NO: 88.

26. The fusion protein of claim 24, wherein the fusion protein comprises the sequence of SEQ ID NO: 90.

27. The fusion protein of claim 24, wherein the fusion protein comprises the sequence of SEQ ID NO: 99.

28. The fusion protein of claim 24, wherein the fusion protein comprises the sequence of SEQ ID NO: 100.

29. The fusion protein of claim 1, further comprising a targeting reagent.

30. The fusion protein of claim 1, further comprising an epitope.

31. The fusion protein of claim 30, wherein the epitope is a polypeptide selected from the group consisting of SEQ ID NOs: 77-83.

32. The fusion protein of claim 1, further comprising a cell-penetrating agent.

33. The fusion protein of claim 32, wherein the cell-penetrating agent comprises a peptide sequence selected from the group consisting of SEQ ID NOs: 84-87.

34. The fusion protein of claim 1, further comprising a signal sequence.

35. The fusion protein of claim 34, wherein the signal sequence comprises the peptide sequence selected from the group consisting of SEQ ID NOs: 102-104.

36. The fusion protein of claim 1, which is capable of reducing misfolding of α-synuclein proteins in a cell.

37. The fusion protein of claim 1, which is capable of reducing phosphorylated α-synuclein proteins in a cell.

38. The fusion protein of claim 1, which is capable of reducing secretion of α-synuclein proteins.

39. The fusion protein of claim 1, which is capable of reducing α-synuclein-mediated cytotoxicity.

40. A nucleic acid sequence encoding the fusion protein of claim 1-39.

41. The nucleic acid sequence of claim 40, wherein said nucleic acid is DNA.

42. The nucleic acid sequence of claim 40, wherein said nucleic acid is RNA.

43. The nucleic acid sequence of claim 40, wherein said nucleic acid comprises at least one modified nucleic acid.

44. The nucleic acid sequence of claim 40, further comprising a promoter region, 5′ UTR, and 3′ UTR, such as poly(A) signal.

45. The nucleic acid sequence of claim 44, wherein the promoter region comprises a sequence selected from the group consisting of a CMV enhancer sequence, a CMV promoter, a CBA promoter, UBC promoter, GUSB promoter, NSE promoter, Synapsin promoter, MeCP2 promoter and GFAP promoter.

46. A vector comprising the nucleic acid sequence of claim 40-45.

47. The vector of claim 46, wherein the vector is selected from the group consisting of adeno-associated virus (AAV), adenovirus, lentivirus, retrovirus, herpesvirus, poxvirus (vaccinia or myxoma), paramyxovirus (measles, RSV or Newcastle disease virus), baculovirus, reovirus, alphavirus, and flavivirus.

48. A virus particle comprising a capsid and the vector of claim 46 or claim 47.

49. The virus particle of claim 48, wherein the capsid is selected from the group consisting of AAV1, AAV2, AAV3, AAV4, AAV5, AAV6, AAV7, AAV8, AAV9, AAV10 AAV11, AAV12, pseudotyped AAV, a rhesus-derived AAV, AAVrh8, AAVrh10 and AAV-DJan AAV capsid mutant, an AAV hybrid serotype, an organ-tropic AAV, a cardiotropic AAV, and a cardiotropic AAVM41 mutant.

50. The virus particle of claim 49, wherein the capsid is selected from the group consisting of AAV2, AAV5, AAV8, AAV9 and AAVrh10.

51. The virus particle of claim 50, wherein the capsid is AAV2.

52. The virus particle of claim 50, wherein the capsid is AAV5.

53. The virus particle of claim 50, wherein the capsid is AAV8.

54. The virus particle of claim 50, wherein the capsid is AAV9.

55. The virus particle of claim 50, wherein the capsid is AAV rh10.

56. A pharmaceutical composition comprising an agent selected from the group consisting of the fusion protein of claim 1-39, a cell expressing the fusion protein of any of claim 1-39, the nucleic acid of claim 40-45, the vector of claim 46-47, the virus particle of claim 48-55, and a pharmaceutically acceptable carrier or excipient.

57. A method of reducing toxicity of a α-synuclein protein in a cell, comprising contacting said cell with an effective amount of one or more agents selected from the group consisting of the fusion protein of claim 1-39, a cell expressing the fusion protein of any of claim 1-39, the nucleic acid of claim 40-45, the vector of any one of claim 46-47, the virus particle of claim 48-55, and the pharmaceutical composition of claim 56.

58. The method of claim 57, wherein the cell is in a subject.

59. The method of claim 58, wherein the subject is a human.

60. The method of claim 59, wherein the cell is a cell of the central nervous system.

61. The method of claim 60, wherein the subject is identified as having an α-synuclein disease.

62. The method of claim 61, wherein the α-synuclein disease is selected from the group consisting of PD, dementia with Lewy bodies, multiple system atrophy, and diseases related to abnormal accumulation of aggregated α-synuclein proteins (synucleinopathies).

63. The method of claim 62, wherein the α-synuclein disease is PD.

64. The method of claim 63, wherein there is a reduction in the amount of misfolded α-synuclein protein in the cell when compared with a control cell.

65. A method of treating, preventing, or delaying the progression of a α-synuclein disease in a subject in need thereof, the method comprising administering an effective amount of one or more agents selected from the group consisting of with the fusion protein of claim 1-39, a cell expressing the fusion protein of any of claim 1-39, the nucleic acid of claim 40-45, the vector of claim 46-47, the virus particle of claim 48-55, and the pharmaceutical composition of claim 56.

66. The method of claim 65, wherein the α-synuclein disease is selected from the group consisting of PD, dementia with Lewy bodies, multiple system atrophy, and diseases related to abnormal accumulation of aggregated α-synuclein proteins (synucleinopathies).

67. The method of claim 66, wherein the α-synuclein disease is PD.

71. The fusion protein of claim 6, wherein the J domain comprises the sequence of SEQ ID NO: 49.


SEQUENCE LISTING (106 sequences)

SEQ ID NO: 1 - protein, 63 aa, Artificial
  DNAJA1
TYYDVLGVKPNATQEELKKAYRKLALKYHPDKNPNEGEKFKQISQAYEVLSDAKKRELYDKGG

SEQ ID NO: 2 - protein, 63 aa, Artificial
  DNAJA2
KLYDILGVPPGASENELKKAYRKLAKEYHPDKNPNAGDKFKEISFAYEVLSNPEKRELYDRYG

SEQ ID NO: 3 - protein, 66 aa, Artificial
  DNAJA3
DYYQILGVPRNASQKEIKKAYYQLAKKYHPDTNKDDPKAKEKFSQLAEAYEVLSDEVKRKQYDAYG

SEQ ID NO: 4 - protein, 67 aa, Artificial
  DNAJA4
ETQYYDILGVKPSASPEEIKKAYRKLALKYHPDKNPDEGEKFKLISQAYEVLSDPKKRDVYDQGGEQ

SEQ ID NO: 5 - protein, 69 aa, Artificial
  DNAJB1
GKDYYQTLGLARGASDEEIKRAYRRQALRYHPDKNKEPGAEEKFKEIAEAYDVLSDPRKREIFDRYGEE

SEQ ID NO: 6 - protein, 70 aa, Artificial
  DNAJB2
ASYYEILDVPRSASADDIKKAYRRKALQWHPDKNPDNKEFAEKKFKEVAEAYEVLSDKHKREIYDRYGRE

SEQ ID NO: 7 - protein, 69 aa, Artificial
  DNAJB3
MVDYYEVLDVPRQASSEAIKKAYRKLALKWHPDKNPENKEEAERRFKQVAEAYEVLSDAKKRDIYDRYG

SEQ ID NO: 8 - protein, 69 aa, Artificial
  DNAJB4
GKDYYCILGIEKGASDEDIKKAYRKQALKFHPDKNKSPQAEEKFKEVAEAYEVLSDPKKREIYDQFGEE

SEQ ID NO: 9 - protein, 65 aa, Artificial
  DNAJB5
DYYKILGIPSGANEDEIKKAYRKMALKYHPDKNKEPNAEEKFKEIAEAYDVLSDPKKRGLYDQYG

SEQ ID NO: 10 - protein, 68 aa, Artificial
  DNAJB6
VDYYEVLGVQRHASPEDIKKAYRKLALKWHPDKNPENKEEAERKFKQVAEAYEVLSDAKKRDIYDKYG

SEQ ID NO: 11 - protein, 67 aa, Artificial
  DNAJB7
DYYEVLGLQRYASPEDIKKAYHKVALKWHPDKNPENKEEAERKFKEVAEAYEVLSNDEKRDIYDKYG

SEQ ID NO: 12 - protein, 67 aa, Artificial
  DNAJB8
NYYEVLGVQASASPEDIKKAYRKLALRWHPDKNPDNKEEAEKKFKLVSEAYEVLSDSKKRSLYDRAG

SEQ ID NO: 13 - protein, 65 aa, Artificial
  DNAJB9
SYYDILGVPKSASERQIKKAFHKLAMKYHPDKNKSPDAEAKFREIAEAYETLSDANRRKEYDTLG

SEQ ID NO: 14 - protein, 66 aa, Artificial
  DNAJB11
DFYKILGVPRSASIKDIKKAYRKLALQLHPDRNPDDPQAQEKFQDLGAAYEVLSDSEKRKQYDTYG

SEQ ID NO: 15 - protein, 65 aa, Artificial
  DNAJB12
YEILGVSRGASDEDLKKAYRRLALKFHPDKNHAPGATEAFKAIGTAYAVLSNPEKRKQYDQFGDD

SEQ ID NO: 16 - protein, 65 aa, Artificial
  DNAJB13
DYYSVLGITRNSEDAQIKQAYRRLALKHHPLKSNEPSSAEIFRQIAEAYDVLSDPMKRGIYDKFG

SEQ ID NO: 17 - protein, 65 aa, Artificial
  DNAJB14
NYYEVLGVTKDAGDEDLKKAYRKLALKFHPDKNHAPGATDAFKKIGNAYAVLSNPEKRKQYDLTG

SEQ ID NO: 18 - protein, 65 aa, Artificial
  DNAJC1
NFYQFLGVQQDASSADIRKAYRKLSLTLHPDKNKDENAETQFRQLVAIYEVLKDDERRQRYDDIL

SEQ ID NO: 19 - protein, 74 aa, Artificial
  DNAJC2
DHYAVLGLGHVRYKATQRQIKAAHKAMVLKHHPDKRKAAGEPIKEGDNDYFTCITKAYEMLSDPVKRRAFNSVD

SEQ ID NO: 20 - protein, 69 aa, Artificial
  DNAJC3
DYYKILGVKRNAKKQEIIKAYRKLALQWHPDNFQNEEEKKKAEKKFIDIAAAKEVLSDPEMRKKFDDGE

SEQ ID NO: 21 - protein, 66 aa, Artificial
  DNAJC4
TYYELLGVHPGASTEEVKRAFFSKSKELHPDRDPGNPSLHSRFVELSEAYRVLSREQSRRSYDDQL

SEQ ID NO: 22 - protein, 70 aa, Artificial
  DNAJC5
GESLYHVLGLDKNATSDDIKKSYRKLALKYHPDKNPDNPEAADKFKEINNAHAILTDATKRNIYDKYGSL

SEQ ID NO: 23 - protein, 66 aa, Artificial
  DNAJC5B
ALYEILGLHKGASNEEIKKTYRKLALKHHPDKNPDDPAATEKFKEINNAHAILTDISKRSIYDKYG

SEQ ID NO: 24 - protein, 65 aa, Artificial
  DNAJC6
TKWKPVGMADLVTPEQVKKVYRKAVLVVHPDKATGQPYEQYAKMIFMELNDAWSEFENQGQKPLY

SEQ ID NO: 25 - protein, 71 aa, Artificial
  DNAJC7
DYYKILGVDKNASEDEIKKAYRKRALMHHPDRHSGASAEVQKEEEKKFKEVGEAFTILSDPKKKTRYDSGQ

SEQ ID NO: 26 - protein, 68 aa, Artificial
  DNAJC8
NPFEVLQIDPEVTDEEIKKRFRQLSILVHPDKNQDDADRAQKAFEAVDKAYKLLLDQEQKKRALDVIQ

SEQ ID NO: 27 - protein, 68 aa, Artificial
  DNAJC9
DLYRVLGVRREASDGEVRRGYHKVSLQVHPDRVGEGDKEDATRRFQILGKVYSVLSDREQRAVYDEQG

SEQ ID NO: 28 - protein, 66 aa, Artificial
  DNAJC10
DFYSLLGVSKTASSREIRQAFKKLALKLHPDKNPNNPNAHGDFLKINRAYEVLKDEDLRKKYDKYG

SEQ ID NO: 29 - protein, 69 aa, Artificial
  DNAJC11
DYYSLLNVRREASSEELKAAYRRLCMLYHPDKHRDPELKSQAERLFNLVHQAYEVLSDPQTRAIYDIYG

SEQ ID NO: 30 - protein, 66 aa, Artificial
  DNAJC12
DYYTLLGCDELSSVEQILAEFKVRALECHPDKHPENPKAVETFQKLQKAKEILTNEESRARYDHWR

SEQ ID NO: 31 - protein, 66 aa, Artificial
  DNAJC13
DAYEVLNLPQGQGPHDESKIRKAYFRLAQKYHPDKNPEGRDMFEKVNKAYEFLCTKSAKIVDGPDP

SEQ ID NO: 32 - protein, 65 aa, Artificial
  DNAJC14
NPFHVLGVEATASDVELKKAYRQLAVMVHPDKNHHPRAEEAFKVLRAAWDIVSNAEKRKEYEMKR

SEQ ID NO: 33 - protein, 55 aa, Artificial
  DNAJC15
EAGLILGVSPSAGKAKIRTAHRRVMILNHPDKGGSPYVAAKINEAKDLLETTTKH

SEQ ID NO: 34 - protein, 65 aa, Artificial
  DNAJC16
DPYRVLGVSRTASQADIKKAYKKLAREWHPDKNKDPGAEDKFIQISKAYEILSNEEKRSNYDQYG

SEQ ID NO: 35 - protein, 66 aa, Artificial
  DNAJC17
DLYALLGIEEKAADKEVKKAYRQKALSCHPDKNPDNPRAAELFHQLSQALEVLTDAAARAAYDKVR

SEQ ID NO: 36 - protein, 65 aa, Artificial
  DNAJC18
NYYEILGVSRDASDEELKKAYRKLALKFHPDKNCAPGATDAFKAIGNAFAVLSNPDKRLRYDEYG

SEQ ID NO: 37 - protein, 55 aa, Artificial
  DNAJC19
EAALILGVSPTANKGKIRDAHRRIMLLNHPDKGGSPYIAAKINEAKDLLEGQAKK

SEQ ID NO: 38 - protein, 72 aa, Artificial
  DNAJC20
DYFSLMDCNRSFRVDTAKLQHRYQQLQRLVHPDFFSQRSQTEKDFSEKHSTLVNDAYKTLLAPLSRGLYLLK

SEQ ID NO: 39 - protein, 67 aa, Artificial
  DNAJC21
CHYEALGVRRDASEEELKKAYRKLALKWHPDKNLDNAAEAAEQFKLIQAAYDVLSDPQERAWYDNHR

SEQ ID NO: 40 - protein, 65 aa, Artificial
  DNAJC22
LAYQVLGLSEGATNEEIHRSYQELVKVWHPDHNLDQTEEAQRHFLEIQAAYEVLSQPRKPWGSRR

SEQ ID NO: 41 - protein, 62 aa, Artificial
  DNAJC23
NPYEVLNLDPGATVAEIKKQYRLLSLKYHPDKGGDEVMFMRIAKAYAALTDEESRKNWEEFG

SEQ ID NO: 42 - protein, 72 aa, Artificial
  DNAJC24
DWYSILGADPSANISDLKQKYQKLILMYHPDKQSTDVPAGTVEECVQKFIEIDQAWKILGNEETKREYDLQR

SEQ ID NO: 43 - protein, 76 aa, Artificial
  DNAJC25
DCYEVLGVSRSAGKAEIARAYRQLARRYHPDRYRPQPGDEGPGRTPQSAEEAFLLVATAYETLKDEETRKDYDYML

SEQ ID NO: 44 - protein, 65 aa, Artificial
  DNAJC26
SRWTPVGMADLVAPEQVKKHYRRAVLAVHPDKAAGQPYEQHAKMIFMELNDAWSEFENQGSRPLF

SEQ ID NO: 45 - protein, 57 aa, Artificial
  DNAJC27
DSWDMLGVKPGASRDEVNKAYRKLAVLLHPDKCVAPGSEDAFKAVVNARTALLKNIK

SEQ ID NO: 46 - protein, 65 aa, Artificial
  DNAJC28
EYYRLLNVEEGCSADEVRESFHKLAKQYHPDSGSNTADSATFIRIEKAYRKVLSHVIEQTNASQS

SEQ ID NO: 47 - protein, 88 aa, Artificial
  DNAJC29
ILKEVTSVVEQAWKLPESERKKIIRRLYLKWHPDKNPENHDIANEVFKHLQNEINRLEKQAFLDQNADRASRRTFSTSASRFQSDKYS

SEQ ID NO: 48 - protein, 66 aa, Artificial
  DNAJC30
ALYDLLGVPSTATQAQIKAAYYRQCFLYHPDRNSGSAEAAERFTRISQAYVVLGSATLRRKYDRGL

SEQ ID NO: 49 - protein, 64 aa, Artificial
  SV40 Jdomain
QLMDLLGLERSAWGNIPLMRKAYLKKCKEFHPDKGGDEEKMKKMNTLYKKMEDGVKYAHQPDFG

SEQ ID NO: 50 - protein, 70 aa, Artificial
  Bacterial J-domain
KQDYYEILGVSKTAEEREIRKAYKRLAMKYHPDRNQGDKEAEAKFKEIKEAYEVLTDSQKRAAYDQYGHA

SEQ ID NO: 51 - protein, 10 aa, Artificial
  SynBP1
KDGIVNGVKA

SEQ ID NO: 52 - protein, 7 aa, Artificial
  SynBP2
WRQTRKD

SEQ ID NO: 53 - protein, 7 aa, Artificial
  SynBP3
HYAKNPI

SEQ ID NO: 54 - protein, 7 aa, Artificial
  SynBP4
ATINKSL

SEQ ID NO: 55 - protein, 7 aa, Artificial
  SynBP5
RRRGMAI

SEQ ID NO: 56 - protein, 7 aa, Artificial
  SynBP6
TKHGPRK

SEQ ID NO: 57 - protein, 7 aa, Artificial
  SynBP7
SLKRLPK

SEQ ID NO: 58 - protein, 7 aa, Artificial
  SynBP8
RLRGRNQ

SEQ ID NO: 59 - protein, 7 aa, Artificial
  SynBP9
WPFHHHR

SEQ ID NO: 60 - protein, 7 aa, Artificial
  SynBP10
HLYHHKT

SEQ ID NO: 61 - protein, 7 aa, Artificial
  SynBP11
THIHHPS

SEQ ID NO: 62 - protein, 7 aa, Artificial
  SynBP12
MMMMMRL

SEQ ID NO: 63 - protein, 248 aa, Artificial
  NAC32 (intrabody)
QVQLQQSGPGLVKPSQTLSLTCAISGDSVSSNSAAWNWIRQSPSRGLEWLGRTFYRSKWYNDYAASVKSRITIDPDTSKNQFSLQLNSVTPEDTAVYYCTRQNLAGPFDSWGQGTLVTVSSGILGSGGGGSGGGGSGGGGSEIVMTQSPGTLSLSPGERATLSCRASQSVSSNYLAWYQQKAGQAPRLLISGASSRATGIPDRFSGSGSGTDFTLTISRLEPEDFAVYYCQQYGSSTAFGPGTKVDIK

SEQ ID NO: 64 - protein, 247 aa, Artificial
  Syn_scFv
LQSVLTQPPSVSGAPGQRVAISCTGTSSDIGTGYDVNWYQHLPGTAPRLLIYGNTYRPSGVPDRFSASTGSKSGTSASLVITDLQAEDEGDYYCQSFDNSLRGSVFGGGTKVTVLGEGKSSGSGSESKASEVQLVQSGGGVVKPGGSLRLSCEASGFILSDYYMTWIRQAPGKGLEWLAVIDITSSYTNYADSVKGRFTISRDNAKNSVYLQMNSLRAEDTAVYYCARLESGFFDYWGQGTLVTVSS

SEQ ID NO: 65 - protein, 11 aa, Artificial
  QBP1
SNWKWWPGIFD

SEQ ID NO: 66 - skipped

SEQ ID NO: 67 - protein, 4 aa, Artificial
  GTGS
GTGS

SEQ ID NO: 68 - protein, 5 aa, Artificial
  GLESR
GLESR

SEQ ID NO: 69 - protein, 4 aa, Artificial
  GGSG
GGSG

SEQ ID NO: 70 - protein, 5 aa, Artificial
  GGGGS
GGGGS

SEQ ID NO: 71 - protein, 5 aa, Artificial
  DIAAA
DIAAA

SEQ ID NO: 72 - protein, 5 aa, Artificial
  EAAAK
EAAAK

SEQ ID NO: 73 - protein, 15 aa, Artificial
  GGGGSGGGGSGGGGS
GGGGSGGGGSGGGGS

SEQ ID NO: 74 - protein, 11 aa, Artificial
  AEAAAKEAAAK
AEAAAKEAAAK

SEQ ID NO: 75 - protein, 15 aa, Artificial
  SGGGSGGGGSGGGGS
SGGGSGGGGSGGGGS

SEQ ID NO: 76 - protein, 25 aa, Artificial
  DIGGGGSGGGGSGGGGSGGGGSAAA
DIGGGGSGGGGSGGGGSGGGGSAAA

SEQ ID NO: 77 - protein, 232 aa, Artificial
  Human IgG1 Fc domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 8 aa, Artificial
  FLAG epitope
DYKDDDDK

SEQ ID NO: 79 - protein, 6 aa, Artificial
  His6
HHHHHH

SEQ ID NO: 80 - protein, 10 aa, Artificial
  c-myc
EQKLISEEDL

SEQ ID NO: 81 - protein, 9 aa, Artificial
  HA
YPYDVPDYA

SEQ ID NO: 82 - protein, 14 aa, Artificial
  V5 epitope
GKPIPNPLLGLDST

SEQ ID NO: 83 - protein, 220 aa, Artificial
  Glutathione-S-transferase
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 84 - protein, 9 aa, Artificial
  RKKRRQRRR
RKKRRQRRR

SEQ ID NO: 85 - protein, 15 aa, Artificial
  RQIKWFQNRRMKWKK
RQIKWFQNRRMKWKK

SEQ ID NO: 86 - protein, 21 aa, Artificial
  KETWWETWWTEWSQPKKKRKV
KETWWETWWTEWSQPKKKRKV

SEQ ID NO: 87 - protein, 17 aa, Artificial
  CSIPPEVKFNKPFVYLI
CSIPPEVKFNKPFVYLI

SEQ ID NO: 88 - protein, 110 aa, Artificial
  JB1-SynBP1
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAKDGIVNGVKA

SEQ ID NO: 89 - protein, 110 aa, Artificial
  J(P33Q)-SynBP1
MGKDYYQTLGLARGASDEEIKRAYRRQALRYHQDKNKEPGAEEKFKEIAEAYDVLSDPRKREIFDRYGEEGLKGSDIGGGGSGGGGSGGGGSGGGGSAAAKDGIVNGVKA

SEQ ID NO: 90 - protein, 120 aa, Artificial
  JB1-2XSynBP1
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAKDGIVNGVKAKDGIVNGVKA

SEQ ID NO: 91 - protein, 123 aa, Artificial
  SynBP1-JB1-SynBP1
MKDGIVNGVKAEFMGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAKDGIVNGVKA

SEQ ID NO: 92 - protein, 90 aa, Artificial
  SynBP2-JB1
MWRQTRKDEFMGKDYYQTLGLARGASDEEIKRAYRRQALRYHPDKNKEPGAEEKFKEIAEAYDVLSDPRKREIFDRYGEEGLKGSDIAAA

SEQ ID NO: 93 - protein, 111 aa, Artificial
  JB6-SynBP1
MVDYYEVLGVQRHASPEDIKKAYRKLALKWHPDKNPENKEEAERKFKQVAEAYEVLSDAKKRDIYDKYGKEGLNGGDIGGGGSGGGGSGGGGSGGGGSAAAKDGIVNGVKA

SEQ ID NO: 94 - protein, 110 aa, Artificial
  JB13-SynBP1
MGQDYYSVLGITRNSEDAQIKQAYRRLALKHHPLKSNEPSSAEIFRQIAEAYDVLSDPMKRGIYDKFGEEGLKGGDIGGGGSGGGGSGGGGSGGGGSAAAKDGIVNGVKA

SEQ ID NO: 95 - protein, 348 aa, Artificial
  JB1-NAC32
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAAQVQLQQSGPGLVKPSQTLSLTCAISGDSVSSNSAAWNWIRQSPSRGLEWLGRTFYRSKWYNDYAASVKSRITIDPDTSKNQFSLQLNSVTPEDTAVYYCTRQNLAGPFDSWGQGTLVTVSSGILGSGGGGSGGGGSGGGGSEIVMTQSPGTLSLSPGERATLSCRASQSVSSNYLAWYQQKAGQAPRLLISGASSRATGIPDRFSGSGSGTDFTLTISRLEPEDFAVYYCQQYGSSTAFGPGTKVDIK

SEQ ID NO: 96 - protein, 347 aa, Artificial
  JB1-Syn_scFv
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAALQSVLTQPPSVSGAPGQRVAISCTGTSSDIGTGYDVNWYQHLPGTAPRLLIYGNTYRPSGVPDRFSASTGSKSGTSASLVITDLQAEDEGDYYCQSFDNSLRGSVFGGGTKVTVLGEGKSSGSGSESKASEVQLVQSGGGVVKPGGSLRLSCEASGFILSDYYMTWIRQAPGKGLEWLAVIDITSSYTNYADSVKGRFTISRDNAKNSVYLQMNSLRAEDTAVYYCARLESGFFDYWGQGTLVTVSS

SEQ ID NO: 97 - protein, 111 aa, Artificial
  JB1-QBP1
MGKDYYQTLGLARGASDEEIKRAYRRQALRYHPDKNKEPGAEEKFKEIAEAYDVLSDPRKREIFDRYGEEGLKGSDIGGGGSGGGGSGGGGSGGGGSAAASNWKWWPGIFD

SEQ ID NO: 98 - protein, 90 aa, Artificial
  JB1GGGS_SynBP1
MGKDYYQTLGLARGASDEEIKRAYRRQALRYHPDKNKEPGAEEKFKEIAEAYDVLSDPRKREIFDRYGEEGLKGSGGGGSKDGIVNGVKA

SEQ ID NO: 99 - protein, 90 aa, Artificial
  JB1EAAAK-SynBP1
MGKDYYQTLGLARGASDEEIKRAYRRQALRYHPDKNKEPGAEEKFKEIAEAYDVLSDPRKREIFDRYGEEGLKGSEAAAKKDGIVNGVKA

SEQ ID NO: 100 - protein, 85 aa, Artificial
  JB1SynBP1
MGKDYYQTLGLARGASDEEIKRAYRRQALRYHPDKNKEPGAEEKFKEIAEAYDVLSDPRKREIFDRYGEEGLKGSKDGIVNGVKA

SEQ ID NO: 101 - protein, 91 aa, Artificial
  JB1GGGGS-QBP1
MGKDYYQTLGLARGASDEEIKRAYRRQALRYHPDKNKEPGAEEKFKEIAEAYDVLSDPRKREIFDRYGEEGLKGSGGGGSSNWKWWPGIFD

SEQ ID NO: 102 - protein, 17 aa, Artificial
  MGVKVLFALICIAVAEA
MGVKVLFALICIAVAEA

SEQ ID NO: 103 - protein, 19 aa, Artificial
  MAPVQLLGLLVLFLPAMRC
MAPVQLLGLLVLFLPAMRC

SEQ ID NO: 104 - protein, 19 aa, Artificial
  MAVLGLLFCLVTFPSCVLS
MAVLGLLFCLVTFPSCVLS

SEQ ID NO: 105 - protein, 140 aa, Artificial
  alpha-synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 106 - protein, 140 aa, Artificial
  alpha-synuclein (A53T)
MDVFMKGLSKAKEGVVAAAEKTKQGVAEAAGKTKEGVLYVGSKTKEGVVHGVTTVAEKTKEQVTNVGGAVVTGVTAVAQKTVEGAGSIAAATGFVKKDQLGKNEEGAPQEGILEDMPVDPDNEAYEMPSEEGYQDYEPEA